(19) United States
(12) Patent Application Publication
(10) Pub. No.: US 2003/0186419 A1
(43) Pub. Date: Oct. 2, 2003

(54) Masp-3, a complement-fixing enzyme, and uses for it

(76) Inventor: Jens Christian Jensenius, Odense M (DK)

Correspondence Address: BROWDY AND NEIMARK, P.L.L.C., 624 NINTH STREET, NW, SUITE 300, WASHINGTON, DC, 20001-5303, US

(21) Appl. No.: 10/148,671

(22) PCT Filed: Nov. 30, 2000
(86) PCT No.: PCT/DK00/00659

(51) Int. Cl. C12N 9/64; C07H 21/04; C12P 21/02; C12N 5/06

(52) U.S. Cl. 435/226; 435/69.1; 435/320.1; 435/325; 536/23.2

(57) ABSTRACT

The invention relates to the discovery and characterization of mannan binding lectin-associated serine protease 3 (MASP-3), a new serine protease that acts in the MBLectin complement fixation pathway.

### FIELD OF THE INVENTION

The invention is in the general field of innate immune defence and the pathways for complement fixation involving mannan-binding lectin (MBL), also termed mannan binding protein or mannose-binding protein (MBP).

### BACKGROUND OF THE INVENTION

The complement system comprises a complex array of enzymes and non-enzymatic proteins of importance to the function of the innate as well as the adaptive immune defense¹. Until recently two modes of activation were known, the classical pathway initiated by antibody-antigen complexes and the alternative pathway initiated by certain structures on microbial surfaces. A third, novel antibody-independent pathway of complement activation has been described². This pathway is initiated when mannan-binding lectin (MBL, first described as mannan-binding proteins, MBP, see Ezekowitz, U.S. Pat. No. 5,270,199) binds to carbohydrates.

MBL is structurally related to the C1q subcomponent of component C1 of complement, and it appears that MBL activates the complement system via an associated serine protease termed MASP⁴ or p100⁵, which is similar to the C1r and C1s components of the classical pathway. The new complement activation pathway is called the MBLectin pathway. According to the mechanism postulated for this pathway, MBL binds to specific carbohydrate structures found on the surface of a range of microorganisms including bacteria, yeast, parasitic protozoa and viruses⁶, and its antimicrobial activity results from activation of the terminal, lytic complement pathway components⁷ or promoting phagocytosis⁸.

Reportedly, the level of MBL in plasma may be genetically determined^{9,10,11}. MBL deficiency is associated with susceptibility to frequent infections with a variety of microorganisms in childhood^{12,13}, and, possibly, in adults^{15,16}. Recent information associates MBL deficiency with HIV infection and with more rapid death following development of AIDS^{15,16}. MBL binds to the a galactosyl form of IgG (G0), which is found at elevated concentrations in rheumatoid arthritis patients, and then activates complement¹⁷. MBL deficiency is also associated with a predisposition to recurrent spontaneous abortions¹⁸, and also to development of systemic lupus erythrematosus¹⁹.

In the first clinical reconstitution trial, an infant MBL-deficient girl suffering from recurrent infections was apparently cured by injections With purified MBL²⁰. For a recent review on MBL, see ref. 6.

Relatively high frequencies of MBL mutations associated with MBL-deficiency have been reported in all populations studied. This observation has led to the hypothesis that MBL may, in certain cases, render the individual more susceptible to certain intracellular infectious agents exploiting MBL to gain access to the target tissues²¹. Since MBL is a very powerful activator of the complement system, it may also be that inexpedient activation through microbial carbohydrates or endotoxins can lead to damaging inflammatory responses¹⁰. Thus, the overall survival of a population may benefit from the wide individual range of MBL concentrations.

MASP-1 (MBL-associated serine protease 1) is a serine protease similar in structure to C1r and C1s of the complement pathway although it has a histidine loop structure of the type found in trypsin and trypsin-like serine proteases. MASP-1 has been found to be involved in complement activation by MBL. A cDNA clone encoding MASP-1 has been reported that encodes a putative leader peptide of 19 amino acids followed by 680 amino acid residues predicted to form the mature peptide.

MASP-2 (MBL-associated serine protease 2)²² is a serine protease similar in structure to C1r and C1s of the complement pathway. Like these, and contrary to MASP-1, it has no histidine loop structure of the type found in trypsin and trypsin-like serine proteases. MASP-2 has been found to be involved in complement activation by MBL.

### SUMMARY OF THE INVENTION

The invention relates to the isolation and characterization of a lectin associated serine protease (MASP-3). MASP-3 shows some homology with the previously reported MASPs (MASP-1 and MASP-2) and the two C1q-associated serine proteases, C1r and C1s.

We have purified MASP-3 and characterized it by its association with lectin, its molecular size and its partial amino acid sequence. We have cloned a cDNA fragment and determined its base sequence, which translates into an amino acid sequence encompassing some of the sequenced peptides. Like MASP-1 and MASP-2, MASP-3 partially co-purifies with MBL, and is likely to be involved in mediating the biological effects of the MBL complex.

Thus, one aspect of the invention features substantially pure MASP-3 polypeptides and nucleic acids encoding such polypeptides. Preferably, the MASP-3 polypeptide retains one or more MASP-3 functions, such as being capable of associating with mannan-binding lectin (MBL) or/and having serine protease activity, a substantially pure mannan-binding lectin associated serine protease-3 (MASP-3) polypeptide, preferably a polypeptide being capable of associating with mannan-binding lectin (MBL).

Another aspect is the production of anti-MASP-3 antibodies and the use of such antibodies for the construction of assays for MASP-3 and the use of such assays for determining clinical syndroms associated with over or under expression of this protein, such as an antibody produced by administering an MASP-3 polypeptide, or part of the MASP-3 polypeptide, or DNA encoding any such polypeptide, according to claim 1 to an animal with the aim of producing antibody.

Some MASP-3 polypeptides according to the invention, e.g., those used in binding assays, may be conjugated to a label so as to permit detection and/or quantification of their presence in the assay. Suitable labels include enzymes which generate a signal (e.g., visible absorption), fluorophores, radionuclides, etc. Other MASP-3 polypeptides are capable of competitively inhibiting one of the MASP-3 activities, and thereby are useful in evaluating MASP-3 function. Other MASP-3 polypeptides are useful antigens or haptens for producing antibodies as described below. Compounds which competitively inhibit a MASP-3 activity are also featured. Preferably, such compounds act by inhibiting the serine protease activity of MASP-3 or of a fragment of MASP-3. Such compounds may include fragments of MBL or of MASP-3 which competitively inhibit the MBL-MASP-3 interactions critical to the function of the complex.

Specific polypeptides according to this aspect of the invention include: a) a polypeptide with a molecular mass of 48K and containing or comprising the sequence identified as SEQ ID NO:3 (IIGGRNAEPGLFPWQALVV); b) a polypeptide with a molecular mass of approximately 110K and containing or comprising the sequence identified as SEQ ID NO:3; c) a polypeptide encompassing the amino acid sequences identified as SEQ ID NO:4 (WQALIVEDTSRVPNDKWFGSGALLSASWILTAAHVLRSQRRDTTVIPVSKEHVTVYL); d) a polypeptide comprising SEQ ID NO:2 including any functional equivalent thereof; e) a polypeptide comprising the B-chain of MASP-3, corresponding to residues 435 (Glu) to 728 (Arg) of SEQ ID NO:2, including any functional equivalent thereof.

Another aspect of the invention includes an isolated nucleic acid molecule comprising a nucleotide sequence encoding a polypeptide encompassing sequences that are at least 85% identical, such as at least 90% identical, for example at least 95% identical to any of the sequences of SEQ ID NO:1, the coding part of SEQ ID NO:1, i.e. the part of the sequence starting with nucleotide no. 91 (a), and ending with nucleotide no. 2277 (a), and SEQ ID NO:5.

Thus, the invention relates to an isolated nucleic acid molecule encoding the polypeptide according to the invention, the molecule comprising a nucleotide sequence encoding a polypeptide having sequence that is at least 50% identical to the sequence of SEQ ID NO:1, 2, 3 or 5.

The invention also features isolated nucleic acid sequences encoding the above MASP-3 polypeptides. Such nucleic acid sequences may be included in nucleic acid vectors (e.g., expression vectors including those with regulatory nucleic acid elements permitting expression of recombinant nucleic acid in an expression system).

The invention also features isolated nucleic acid sequences encoding polypeptides of the entire 110 kDa MASP-3 protein. Such nucleic acid sequences may be included in nucleic acid vectors (e.g., expression vectors including those with regulatory nucleic acid elements permitting expression of recombinant nucleic acid in an expression system).

The invention also features antibodies that selectively bind to MASP-3. Such antibodies may be made by any of the well known techniques including polyclonal and monoclonal antibody techniques. The antibody may be coupled to a compound comprising a detectable marker, so that it can be used, e.g. in an assay to detect MASP-3.

The polypeptides or antibodies may be formulated into pharmaceutical compositions and administered as therapeutics as described below.

The invention also features methods for detecting MASP-3. The method comprises; obtaining a biological sample, contacting the biological sample with a MASP-3 polypeptide specific binding partner, and detecting the bound complexes, if any, as an indication of the presence of MASP-3 in the biological sample. The binding partner used in the assay may be an antibody, or the assay for MASP-3 may test for complement fixing activity. These assays for MASP-3 may also be used for quantitative assays of MASP-3 or MASP-3 activity in biological samples. One of the binding partners may be specific for MBL thus allowing for the detection of MBL/MASP-3 complexes.

Methods for detecting MASP-3 nucleic acid expression are included in the invention. These methods comprise detecting RNA having a sequence encoding a MASP-3 polypeptide by mixing the sample with a nucleic acid probe that specifically hybridizes under stringent conditions to a nucleic acid sequence encoding all or a fragment of MASP-3.

The invention also features methods for treating patients deficient in MASP-3 or MASP-3 activity. This is accomplished by administering to the patient MASP-3 polypeptide or nucleic acid encoding MASP-3. Because it is sometimes desirable to inhibit MASP-3 activity, the invention includes a method for inhibiting the activity of MASP-3 by administering to the patient a compound that inhibits expression or activity of MASP-3. Inhibition of MASP-3 activity may also be achieved by administering a MASP-3 anti-sense nucleic acid sequence.

The invention features an assay for polymorphisms in the nucleic acid sequence encoding MASP-3. A method of detecting the presence of MASP-3-encoding nucleic acid in a sample is claimed. As an example, the method may include mixing the sample with at least one nucleic acid probe capable of forming a complex with MASP-3-encoding nucleic acid under stringent conditions, and determining whether the probe is bound to sample nucleic acid. The invention thus includes nucleic acid probe capable of forming a complex with MASP-3-encoding nucleic acid under stringent conditions.

The invention features an assay for polymorphisms in the polypeptide sequence comprising MASP-3 or its precursor or MASP-3 regulatory sequences.

MASP-3 assays are useful for the determination of MASP-3 levels and MASP-3 activity in patients suffering from various diseases such as infections, inflammatory diseases and spontaneous recurrent abortion. MASP-3 is useful for the treatment of infections when MASP-3 function is suboptimal, and inhibition of MASP-3 activity is useful for regulation of inflammation and adverse effects caused by activity of MASP-3.

Furthermore, the invention relates to the use of a polypeptide as defined herein for preparation of a pharmaceutical composition.

By “lectin associated serine protease 110” or “MASP-3” is meant the polypeptide or activity called “lectin associated serine protease 110” or any other polypeptide having substantial sequence identity with SEQ ID NO:2.

The terms “protein” and “polypeptide” are used herein to describe any chain of amino acids, regardless of length or post-translational modification (for example, glycosylation or phosphorylation). Thus, the term “MASP-3 polypeptide” includes full-length, naturally occurring MASP-3 protein, as well as recombinantly or synthetically produced polypeptide that corresponds to a full-length naturally occurring MASP-3 polypeptide, or to particular domains or portions of a naturally occurring protein. The term also encompassses mature MASP-3 which has an added amine terminal methionine (which is useful for expression in prokaryotic cells).

The term “purified” as used herein refers to a nucleic acid or peptide that is substantially free of cellular material, viral material, or culture medium when produced by recombinant DNA techniques, or chemical precursors or other chemicals when chemically synthesized.

By “isolated nucleic acid molecule” is meant a nucleic acid molecule that is separated in any way from sequences in the naturally occurring genome of an organism. Thus, the term “isolated nucleic acid molecule” includes nucleic acid molecules which are not naturally occurring, e.g., nucleic acid molecules created by recombinant DNA techniques.

The term “nucleic acid molecule” encompasses both RNA and DNA, including cDNA, genomic DNA, and synthetic (e.g., chemically synthesized) DNA. Where single-stranded, the nucleic acid may be a sense strand or an antisense strand.

The term “MBL/MASP complex” encompasses MBL/MASP-1 complexes, MBL/MASP-2 complexes, MBL/MASP-3 complexes, said complexes optionally comprising further substances. For example “MBL/MASP-2 complex” may also comprise other substances.

The invention also encompasses nucleic acid molecules that hybridize, preferably under stringent conditions, to a nucleic acid molecule encoding an MASP-3 polypeptide (e.g., a nucleic acid molecule having the sequence encoding SEQ ID NO:3, e.g., the cDNA sequence shown in FIG. 5, SEQ ID NO:5 (tggcaggccc tgatagtggt ggaggacact tcgagagtgc caaatgacaagtggtttggg agtggggccc tgctctctgc gtcctggatc ctcacagcag ctcatgtgctgcgctcccag cgtagagaca ccacggtgat accagtctcc aaggagcatg tcaccgtctacctg) or any other part of the entire cDNA encoding the complete MASP-3 sequence. In addition, the invention encompasses nucleic acid molecules that hybridize, preferably under stringent conditions, to a nucleic acid molecule having the sequence of the MASP-3 encoding cDNA contained in a clone. Preferably the hybridizing nucleic acid molecule consists of 400, more preferably 200 nucleotides.

Preferred hybridizing nucleic acid molecules encode an activity possessed by MASP-3, e.g., they bind MBL (or another MASP-3 ligand) or can act as serine proteases.

The invention also features substantially pure or isolated MASP-3 polypeptides, preferably those that correspond to various functional domains of MASP-3, or fragments thereof. The polypeptides of the invention encompass amino acid sequences that are substantially identical to the amino acid sequence shown in FIG. 5, or substantially identical to the amino acid sequence of the entire MASP-3 protein.

The polypeptides of the invention can also be chemically synthesized, synthesized by recombinant technology, or they can be purified from tissues in which they are naturally expressed, according to standard biochemical methods of purification.

Also included in the invention are “functional polypeptides” which possess one or more of the biological functions or activities of MASP-3. These functions or activities are described in detail in the specification. A functional polypeptide is also considered within the scope of the invention if it serves as an antigen for production of antibodies that specifically bind to MASP-3 or fragments (particularly determinant containing fragments) thereof.

The functional polypeptides may contain a primary amino acid sequence that has been modified from those disclosed herein. Preferably these modifications consist of conservative amino acid substitutions, as described herein. The polypeptides may be substituted in any manner designed to promote or delay their catabolism (increase their half-life).

Conservative amino acid substitutions as used herein relate to the substitution of one amino acid (within a predetermined group of amino acids) for another amino acid (within the same group) exhibiting similar or substantially similar characteristics.

Within the meaning of the term conservative amino acid substitution as applied herein, one amino acid may be substituted for another within groups of amino acids characterised by having

i) polar side chains (Asp, Glu, Lys, Arg, His, Asn, Gin, Ser, Thr, Tyr, and Cys,)

ii) non-polar side chains (Gly, Ala, Val, Leu, lie, Phe, Trp, Pro, and Met)

iii) aliphatic side chains (Gly, Ala Val, Leu, lie)

iv) cyclic side chains (Phe, Tyr, Trp, His, Pro)

v) aromatic side chains (Phe, Tyr, Trp)

vi) acidic side chains (Asp, Glu)

vii) basic side chains (Lys, Arg, His)

viii) amide side chains (Asn, Gin)

ix) hydroxy side chains (Ser, Thr)

x) sulphor-containing side chains (Cys, Met), and

xi) amino acids being monoamino-dicarboxylic acids or monoamino-monocarboxylic-monoamidocarboxylic acids (Asp, Glu, Asn, Gin).

When the amino acid sequence comprises a substitution of one amino acid for another, such a substitution may be a conservative amino acid substitution as defined herein above. Fragments of MASP-3 according to the present invention may comprise more than one such substitution, such as e.g. two conservative amino acid substitutions, for example three or four conservative amino acid substitutions, such as five or six conservative amino acid substitutions, for example seven or eight conservative amino acid substitutions, such as from 10 to 15 conservative amino acid substitutions, for example from 15 to 25 conservative amino acid substitution. Substitutions can be made within any one or more groups of predetermined amino acids as listed herein above.

The addition or deletion of an amino acid may be an addition or deletion of from 2 to preferably 10 amino acids, such as from 2 to 8 amino acids, for example from 2 to 6 amino acids, such as from 2 to 4 amino acids. However, additions of more than 10 amino acids, such as additions from 10 to 200 amino acids, are also comprised within the present invention.

It will thus be understood that the invention also pertains to immunogenic composition comprising at least one fragment of MASP-3, including any variants and functional equivalents of such at least one fragment.

The fragment of MASP-3 according to the present invention, including any variants and functional equivalents thereof, may in one embodiment comprise less than 100 amino acid residues, such as less than 95 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues.

Functional equivalency as used in the present invention is according to one preferred embodiment established by means of reference to the corresponding functionality of a predetermined MASP-3 fragment, such as e.g. the fragment comprising or essentially consisting of the B chain of MASP-3, or a full length MASP-3 sequence.

Functional equivalents of a fragment of MASP-3 comprising a predetermined amino acid sequence are defined as stated herein above. One method of determining a sequence of immunogenically active amino acids within a known amino acid sequence has been described by Geysen in U.S. Pat. No. 5,595,915 and is incorporated herein by reference.

A further suitably adaptable method for determining structure and function relationships of peptide fragments is described by U.S. Pat. No. 6,013,478, which is herein incorporated by reference.

Functional equivalents of fragments of MASP-3 will be understood to exhibit amino acid sequences gradually departing from the preferred predetermined sequence including a sequence comprising or essentially consisting of a MASP-3 B-chain, as the number and scope of insertions, deletions and substitutions including conservative substitutions increases. This departure is measured as a reduction in homology between the preferred predetermined sequence and the variant or functional equivalent. All complement activating MASP-3 fragments are included within the scope of this invention, regardless of the degree of homology that they show to a preferred predetermined sequence of MASP-3 including the B chain of MASP-3. The reason for this is that some regions of MASP-3 are most likely readily mutatable, or capable of being completely deleted, without any significant biological effect.

A functional variant obtained by substitution may well exhibit some form or degree of native MASP-3 activity, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity between i) a given MASP-3 fragment capable of eliciting a complement stimulating effect and ii) a preferred predetermined fragment of MASP-3, is not a principal measure of the fragment as a variant or functional equivalent of a preferred, predetermined MASP-3 fragment according to the present invention.

A non-conservative substitution leading to the formation of a functionally equivalent fragment of MASP-3 would for example i) differ substantially in hydrophobicity, for example a hydrophobic residue (Val, lie, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

In a further embodiment the present invention relates to functional equivalents of a preferred predetermined fragment of MASP-3, including the B chain of MASP-3, wherein such equivalents comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittie, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same or other MASP-3 fragments and/or MASP-3 molecules, including dimers or unrelated chemical moieties. Such functional equivalents are prepared by in vivo synthesis or by linkage of functionalities to groups which are found in fragment including at any one or both of the N- and C-termini, by means known in the art.

Oligomers of MASP-3 including dimers including homodimers and heterodimers of fragments of MASP-3 according to the invention are also provided for within the scope of the present invention. MASP-3 functional equivalents and variants can be produced as homodimers or heterodimers with other amino acid sequences or with native MASP-3 sequences.

The terms functional MASP-3 equivalents, MASP-3 variants and MASP-3 derivatives as used herein relate to functional equivalents of a fragment of MASP-3 comprising a predetermined amino acid sequence, and such equivalents, derivatives and variants are defined as:

i) MASP-3 or fragments thereof comprising an amino acid sequence capable of being recognised by an antibody also capable of recognising the predetermined amino acid sequence, and/or

ii) MASP-3 or fragments thereof comprising an amino acid sequence capable of forming an association with a component of the MBL pathway, such as the MBL/MASP-2 complex, wherein said component is also capable of forming an association with the predetermined amino acid sequence, and/or

iii) Fragments of MASP-3 having at least a substantially similar complement activating effect as the fragment of MASP-3 comprising said predetermined amino acid sequence, such as inhibiting cleavage of C4 when bound to a MBL/MASP-2 complex.

Polypeptides or other compounds of interest are said to be “substantially pure” when they are distinct from any naturally occuring composition, and suitable for at least one of the uses proposed herein. While preparations that are only slightly altered with respect to naturally occuring substances may be somewhat useful, more typically, the preparations are at least 10% by weight (dry weight) the compound of interest. Preferably, the preparation is at least 60%, more preferably at least 75%, and most preferably at least 90%, by weight the compound of interest. Purity can be measured by any appropriate standard method, for example, by column chromatography, polyacrylamide gel electrophoresis, or HPLC analysis.

A polypeptide or nucleic acid molecule is “substantially identical” to a reference polypeptide or nucleic acid molecule if it has a sequence that is at least 85%, preferably at least 90%, and more preferably at least 95%, 98%, or 99% identical to the sequence of the reference polypeptide or nucleic acid molecule.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide which is 50% identical to the reference polypeptide over its entire length. Of course, many other polypeptides will meet the same criteria.

In the case of polypeptide sequences which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

For polypeptides, the length of the reference polypeptide sequence will generally be at least 16 amino acids, preferably at least 20 amino acids, more preferably at least 25 amino acids, and most preferably 35 amino acids, 50 amino acids, or 100 amino acids. For nucleic acids, the length of the reference nucleic acid sequence will generally be at least 50 nucleotides, preferably at least 60 nucleotides, more preferably at least 75 nucleotides, and most preferably 100 nucleotides or 300 nucleotides.

Sequence identity can be measured using sequence analysis software (for example, the Sequence Analysis Software Package of the Genetics Computer Group, University of Wisconsin Biotechnology Center, 1710 University Avenue, Madison, Wis. 53705), with the default parameters as specified therein.

The nucleic acid molecules of the invention can be inserted into a vector, as described below, which will facilitate expression of the insert. The nucleic acid molecules and the polypeptides they encode can be used directly as diagnostic or therapeutic agents, or can be used (directly in the case of the polypeptide or indirectly in the case of a nucleic acid molecule) to generate antibodies that, in turn, are clinically useful as a therapeutic or diagnostic agent. Accordingly, vectors containing the nucleic acid of the invention, cells transfected with these vectors, the polypeptides expressed, and antibodies generated, against either the entire polypeptide or an antigenic fragment thereof, are among the preferred embodiments.

The invention also features antibodies, e.g., monoclonal, polyclonal, and engineered antibodies, which specifically bind MASP-3. By “specifically binds” is meant an antibody that recognizes and binds to a particular antigen, e.g., the MASP-3 polypeptide of the invention, but which does not substantially recognize or bind to other molecules in a sample, e.g., a biological sample, which includes MASP-3. References to constructs of antibody (or fragment thereof coupled to a compound comprising a detectable marker includes constructs made by any technique, including chemical means or by recombinant techniques.

The invention also features antagonists and agonists of MASP-3 that can inhibit or enhance one or more of the functions or activities of MASP-3, respectively. Suitable antagonists can include small molecules (i.e., molecules with a molecular weight below about 500), large molecules (i.e., molecules with a molecular weight above about 500), antibodies that bind and “neutralize” MASP-3 (as described below), polypeptides which compete with a native form of MASP-3 for binding to a protein, e.g., MBL, and nucleic acid molecules that interfere with transcription of MASP-3 (for example, antisense nucleic acid molecules and ribozymes). Agonists of MASP-3 also include small and large molecules, and antibodies other than “neutralizing” antibodies.

The invention also features molecules which can increase or decrease the expression of MASP-3 (e.g., by influencing, transcription or translation). Small molecules (i.e., molecules with a molecular weight below about 500), large molecules (i.e., molecules with a molecular weight above about 500), and nucleic acid molecules that can be used to inhibit the expression of MASP-3 (for example, antisense and ribozyme molecules) or to enhance their expression (for example, expression constructs that place nucleic acid sequences encoding MASP-3 under the control of a strong promoter system), and transgenic animals that express a MASP-3 transgene.

The invention encompasses methods for treating disorders associated with aberrant expression or activity of MASP-3. Thus, the invention includes methods for treating disorders associated with excessive expression or activity of MASP-3. Such methods entail administering a compound which decreases the expression or activity of MASP-3. The invention also includes methods for treating disorders associated with insufficient expression of MASP-3. These methods entail administering a compound which increases the expression or activity of MASP-3.

By “competitively inhibiting” serine protease activity is meant that, for example, the action of an altered MBL or fragment thereof that can bind to a MASP-3 peptide, reversibly or irreversibly without activating or neutralizing serine protease activity. Conversely, a fragment of MASP-3, e.g., a polypeptide encompassing the N-terminal part of MASP-3, may competitively inhibit the binding of the intact MASP-3 and thus effectively inhibit the activation of MASP-3.

The invention also features methods for detecting a MASP-3 polypeptide. Such methods include: obtaining a biological sample; contacting the sample with an antibody that specifically binds MASP-3 under conditions which permit specific binding; and detecting any antibody-MASP-3-complexes formed.

In addition, the present invention encompasses methods and compositions for the diagnostic evaluation, typing, and prognosis of disorders associated with inappropriate expression or activity of MASP-3. For example, the nucleic acid molecules of the invention can be used as diagnostic hybridization probes to detect, for example, inappropriate expression of MASP-3 or mutations in the MASP-3 gene. Such methods may be used to classify cells by the level of MASP-3 expression.

Alternatively, the nucleic acid molecules can be used as primers for diagnostic PCR analysis for the identification of gene mutations, allelic variations and regulatory defects in the MASP-3 gene. The present invention further provides for diagnostic kits for the practice of such methods.

The invention features methods of identifying compounds that modulate the expression or activity of MASP-3 by assessing the expression or activity of MASP-3 in the presence and absence of a selected compound. A difference in the level of expression or activity of MASP-3 in the presence and absence of the selected compound indicates that the selected compound is capable of modulating expression or activity or MASP-3. Expression can be assessed either at the level of gene expression (e.g., by measuring mRNA) or protein expression by techniques that are well known to skilled artisans. The activity of MASP-3 can be assessed functionally, i.e., by assaying the enzymatic activity of the compound.

The preferred methods and materials are described below in examples which are meant to illustrate, not limit, the invention. Skilled artisans will recognize methods and materials that are similar or equivalent to those described herein, and that can be used in the practice or testing of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the detailed description, and from the claims.

### BRIEF DESCRIPTION OF THE DRAWINGS

FIG. 1 depict a Western blot of human plasma proteins purified by sugar affinity chromatography and developed by anti-pMASP-3 antibody. Lane 1 represent a sample which was reduced prior to electrophoresis whereas lane 2 has been run at non-reducing conditions. The arrows indicate the position of the 48 kDa (reduced) and the 110 kDa (non-reduced) MASP-3 bands.

FIG. 2 represent a result demonstrating molecular complexes formed between MBL and MASP-3. The lectin preparation was incubated in wells coated with monoclonal anti-MBL antibody, monoclonal anti-MASP-1 antibody or, as a negative control, wells coated with non-specific monoclonal immunoglobulin of the same subclass. The lectin preparation was diluted both in calcium containing buffer and in EDTA containing buffer. The proteins captured by the antibody were eluted and analyzed by SDS-PAGE/Western blotting under non-reduced conditions. The blot was developed with anti-pMASP-3 antibody. Lane 1 represents unfractionated lectin preparation. Lanes 2 and 3 represent eluates from wells coated with non-sense IgG and incubated with lectin preparation (lane 2 in the presence of calcium, lane 3 in the presence of EDTA), while lanes 4 and 5 represent eluates from wells coated with monoclonal anti-MASP-1 antibody and incubated with lectin preparation (lane 4 in the presence of calcium, lane 5 in the presence of EDTA) and lane 6 and 7 represents eluates from wells coated with monoclonal anti-MBL antibody and incubated with lectin preparation (lane 6 in the presence of calcium, lane 7 in the presence of EDTA). The position of the 110 kDa MASP-3 band is indicated on the figure.

FIG. 3 depict a western blot of human plasma proteins purified on mannose-TSK beads from MBL-deficient serum (Lane 1, reduced and lane 2, non- reduced) or from MBL-deficient serum to which MASP-free MBL has been added (lane 3, reduced and lane 4, non-reduced). The western blot was developed with rat anti-pMASP-3 antibody followed by HRP labelled anti-rat IgG antibody.

FIG. 4 shows the amino acid sequences obtained from the N-terminal part of the 48 kDa MASP-3 band and from peptides obtained from the 48 kDa band MASP-3 band.

FIG. 5 shows the MASP-3 encoding DNA sequence of the PCR product obtained from liver cDNA and deduced partial amino acid sequence.

FIG. 6 shows the sequence alignment of the known amino acid sequences of MASP-3 with those of MASP-2²², MASP-1^{23,24}, C1r^{25,26} and C1s^{27,28}. Identical residues in all four species are indicated by asterisks.

FIG. 7. a, Two-dimensional SDS-PAGE Western blot of MBL complexes purified by affinity chromatography on mannan-Sepharose. The first dimension (horizontal) was run under non-reducing conditions. The lane was reduced and run in the second dimension. The gel was blotted and developed with antibody against the N-terminal peptide of the 42K protein. The second dimension gel was prepared with a separate well for a reduced sample of MBL complexes (lane R), which thus illustrates the pattern after standard one-dimensional electrophoresis. The positions of the M, markers are indicated. b, Association of MASP-3 with MBL. Samples (100 μl) of sera diluted with an equal volume of TBS were incubated in microtitre wells coated with monoclonal anti-MBL antibody, eluted with 100 μl SDS sample buffer for 10 identical wells¹⁹ and examined by SDS-PAGE Western blotting using antibody against the N-terminal peptide of the 42K protein. The samples were: A, normal serum containing MBL 2 μg/ml; B, purified MBL²⁹ (1 μg); D and F, two MBL-deficient sera (MBL concentrations 20 ng/ml); C and E, the same two MBL-deficient sera with MBL added to 2 μg/ml.

FIG. 8. Fractionation of MBL complexes. a, Sucrose gradient centrifugation showing the C4 activating capacity and the MBL content of the fractions. The positions of 7 S IgG and 19 S IgM are indicated. b, SDS-PAGE Western blot of the fractions developed with anti-MBL antibody, c, with anti-MASP-1 antibody²², d, with anti-MASP-2 antibody²⁹, e, with anti-MASP-3 antibody. f, with anti-MASP-2 antibody reacting with MAp19, g, MBL in fractions from ion-exchange chromatography, and h, C3 activating capacities of the same fractions (note the C3α′ chain in lanes 4 and 5).

FIG. 9. The inhibitory activity of MASP-3 on the activation of C4 by MBL complexes. a, dilutions of rMASP-3 (open circles) or control (blocked circles) was incubated with natural MBL complexes for 2 h before adding to mannan-coated microwells. After further overnight incubation at 4° C. and washing of the wells, C4 was added and incubated at 37° C. for 2 h. Activated, bound C4 was quantified with Eu-labelled anti-C4 antibody. Activity (%) was read from a standard curve based on dilutions of MBL complexes. b, rMASP-2³⁰ was mixed with rMBL (to be published) and dilutions of rMASP-3 (open circles) or control (blocked circles), incubated and then added to mannan-coated wells and treated as in a. In the experiments shown (a and b) rMASP-3 was used in the form of culture supernatants of transfected cells with supernatant of sham-transfected cells as control. The same results were obtained with rMASP-3 purified by ion-exchange chromatography.

FIG. 10. a, Deduced amino-acid sequence of the MASP-3 B chain. The sequence (third and fourth lines) is aligned with those of human MASP-1 (NM001879) and MASP-2 (Y09926) B chains (upper two lines), as well as with shark (AB009074) and carp (AB009073) MASP-3 B chains and a partial pig MASP-3 sequence (AW414970) (lower lines). *) identical residues :) conserved substitutions, .) semi-conserved substitutions. The alignment was made with BLOSUM G2 (gap existence cost of 11, residue gap cost of 1, lambda ratio of 0.85). Aligned cysteines are boxed. The cysteines in the histidine loop of MASP-1 are shaded. The three N-glycosylation sites are in bold. b, Genomic organization of the exons encoding MASP-1 and MASP-3. C, Comparison of the protein-encoding regions of the mRNA for MASP-1 and MASP-3.

### DETAILED DESCRIPTION OF THE INVENTION

MASP-3 Nucleic Acid Molecules

The MASP-3 nucleic acid molecules of the invention can be cDNA, genomic DNA, synthetic DNA, or RNA, and can be double-stranded or single-stranded (i.e., either a sense or an antisense strand). Fragments of these molecules are also considered within the scope of the invention, and can be produced, for example, by the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, the polypeptide of SEQ ID NO:5). In addition, these nucleic acid molecules are not limited to sequences that only encode polypeptides, and thus, can include some or all of the non-coding sequences that lie upstream or downstream from a coding sequence.

In a preferred embodiment the invention relates to an isolated nucleic acid molecule encoding the polypeptide defined herein, the molecule comprising a nucleotide sequence encoding a polypeptide having sequence that is at least 50% identical to the sequence of SEQ ID NO:1, 2, 3 or 5. The polypeptide is preferably a mannan-binding lectin associated serine protease-3 (MASP-3) having a polypeptide sequence at least 85% identical to SEQ ID NO:5.

Thus, the isolated nucleic acid sequence preferably encodes a mannan-binding lectin associated serine protease-3 (MASP-3), said nucleic acid sequence being at least 85% identical to SEQ ID NO:4.

The nucleic acid molecules of the invention can be synthesized (for example, by phosphoramidite-based synthesis) or obtained from a biological cell, such as the cell of a mammal. Thus, the nucleic acids can be those of a human, mouse, rat, guinea pig, cow, sheep, horse, pig, rabbit, monkey, dog, or cat. Combinations or modifications of the nucleotides within these types of nucleic acids are also encompassed.

In addition, the isolated nucleic acid molecules of the invention encompass fragments that are not found as such in the natural state. Thus, the invention encompasses recombinant molecules, such as those in which a nucleic acid molecule (for example, an isolated nucleic acid molecule encoding MASP-3) is incorporated into a vector (for example, a plasmid or viral vector) or into the genome of a heterologous cell (or the genome of a homologous cell, at a position other than the natural chromosomal location). Recombinant nucleic acid molecules and uses therefore are discussed further below.

In the event the nucleic acid molecules of the invention encode or act as antisense molecules, they can be used for example, to regulate translation of MASP-3. Techniques associated with detection or regulation of nucleic acid expression are well known to skilled artisans and can be used to diagnose and/or treat disorders associated with MASP-3 activity. These nucleic acid molecules are discussed further below in the context of their clinical utility.

The invention also encompasses nucleic acid molecules that hybridize under stringent conditions to a nucleic acid molecule encoding a MASP-3 polypeptide. The cDNA sequence described herein (SEQ ID NO:3) can be used to identify these nucleic acids, which include, for example, nucleic acids that encode homologous polypeptides in other species, and splice variants of the MASP-3 gene in humans or other mammals. Accordingly, the invention features methods of detecting and isolating these nucleic acid molecules.

Using these methods, a sample (for example, a nucleic acid library, such as a cDNA or genomic library) is contacted (or “screened”) with a MASP-3-specific probe (for example, a fragment of SEQ ID NO:5 that is at least 12 nucleotides long). The probe will selectively hybridize to nucleic acids encoding related polypeptides (or to complementary sequences thereof). Because the polypeptide encoded by MASP-3 is related to other serine ptoteases, the term “selectively hybridize” is used to refer to an event in which a probe binds to nucleic acids encoding MASP-3 (or to complementary sequences thereof) to a detectably greater extent than to nucleic acids encoding other serine proteases (or to complementary sequences thereof. The probe, which can contain at least 12 (for example, 15, 25, 50, 100, or 200 nucleotides) can be produced using any of several standard methods (see, for example, Ausubel et al., “Current Protocols in Molecular Biology, Vol. I,” Green Publishing Associates, Inc., and John Wiley & Sons, Inc., NY, 1989). For example, the probe can be generated using PCR amplification methods in which oligonucleotide primers are used to amplify a MASP-3-specific nucleic acid sequence (for example, a nucleic acid encoding the N-terminus of mature MASP-3) that can be used as a probe to screen a nucleic acid and thereby detect nucleic acid molecules (within the library) that hybridize to the probe.

One single-stranded nucleic acid is said to hybridize to another if a duplex forms between them. This occurs when one nucleic acid contains a sequence that is the reverse and complement of the other (this same arrangement gives rise to the natural interaction between the sense and antisense strands of DNA in the genome and underlies the configuration of the “double helix”). Complete complementarity between the hybridizing regions is not required in order for a duplex to form; it is only necessary that the number of paired bases is sufficient to maintain the duplex under the hybridization conditions used.

In one aspect, the invention relates to a nucleic acid probe capable of forming a complex with MASP-3-encoding nucleic acid under stringent conditions, such as a sequence capable of hybridizing to a nucleic acid sequence identical to SEQ ID NO 5.

The hybridizable probe may be an anti-sense nucleic acid with respect to a nucleic acid sequence encoding MASP-3.

Typically, hybridization conditions are of low to moderate stringency. These conditions favour specific interactions between completely complementary sequences, but allow some non-specific interaction between less than perfectly matched sequences to occur as well. After hybridization, the nucleic acids can be “washed” under moderate or high conditions of stringency to dissociate duplexes that are bound together by some non-specific interaction (the nucleic acids that form these duplexes are thus not completely complementary).

As is known in the art, the optimal conditions for washing are determined empirically, often by gradually increasing the stringency. The parameters that can be changed to affect stringency include, primarily, temperature and salt concentration.

In general, the lower the salt concentration and the higher the temperature, the higher the stringency. Washing can be initiated at a low temperature (for example, room temperature) using a solution containing a salt concentration that is equivalent to or lower than that of the hybridization solution. Subsequent washing can be carried out using progressively warmer solutions having the same salt concentration.

As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where nucleic acids are hybridized, the conditions used to achieve a given level of stringency will vary. There is not one set of conditions, for example, that will allow duplexes to form between all nucleic acids that are 85% identical to one another; hybridization also depends on unique features of each nucleic acid. The length of the sequence, the composition of the sequence (for example, the content of purine-like nucleotides versus the content of pyrimidine-like nucleotides) and the type of nucleic acid (for example, DNA or RNA) affect hybridization. An additional consideration is whether one of the nucleic acids is immobilized (for example, on a filter).

An example of a progression from lower to higher stringency conditions is the following, where the salt content is given as the relative abundance of SSC (a salt solution containing sodium chloride and sodium citrate; 2×SSC is 10-fold more concentrated than 0.2×SSC). Nucleic acids are hybridized at 42° C. in 2×SSC/0.1% SDS (sodium dodecylsulfate; a detergent) and then washed in 0.2×SSC/0.1% SDS at room temperature (for conditions of low stringency); 0.2×SSC/0.1% SDS at 42° C. (for conditions of moderate stringency); and 0.1×SSC at 68° C. (for conditions of high stringency). Washing can be carried out using only one of the conditions given, or each of the conditions can be used (for example, washing for 10-15 minutes each in the order listed above). Any or all of the washes can be repeated. As mentioned above, optimal conditions will vary and can be determined empirically.

A second set of conditions that are considered “stringent conditions” are those in which hybridization is carried out at 50° C. in Church buffer (7% SDS, 0.5% NaHPO₄, 1 M EDTA, 1% bovine serum albumin) and washing is carried out at 50° C. in 2×SSC.

Once detected, the nucleic acid molecules can be isolated by any of a number of standard techniques (see, for example, Sambrook et al., “Molecular Cloning, A Laboratory Manual,” 2nd Ed. Cold Spring Harbor Laboratory Press, Cold Spring Harbor, N.Y., 1989).

The invention also encompasses: (a) expression vectors that contain any of the foregoing MASP-3-related coding sequences and/or their complements (that is, “antisense” sequence); (b) expression vectors that contain any of the foregoing MASP-3-related coding sequences operatively associated with a regulatory element (examples of which are given below) that directs the expression of the coding sequences; (c) expression vectors containing, in addition to sequences encoding a MASP-3 polypeptide, nucleic acid sequences that are unrelated to nucleic acid sequences encoding MASP-3, such as molecules encoding a reporter or marker; and (d) genetically engineered host cells that contain any of the foregoing expression vectors and thereby express the nucleic acid molecules of the invention in the host cell.

Recombinant nucleic acid molecule can contain a sequence encoding a soluble MASP-3, mature MASP-3, MASP-3 having a signal sequence, or functional domains of MASP-3 such as a serine protease domain, a EGF domain, or a MBL-binding domain. The full length MASP-3 polypeptide, a domain of MASP-3, or a fragment thereof may be fused to additional polypeptides, as described below. Similarly, the nucleic acid molecules of the invention can encode the mature form of MASP-3 or a form that encodes a polypeptide which facilitates secretion. In the latter instance, the polypeptide is typically referred to as a proprotein, which can be converted into an active form by removal of the signal sequence, for example, within the host cell.

Proproteins can be converted into the active form of the protein by removal of the inactivating sequence.

The regulatory elements referred to above include, but are not limited to, inducible and non-inducible promoters, enhancers, operators and other elements, which are known to those skilled in the art, and which drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast—mating factors.

Similarly, the nucleic acid can form part of a hybrid gene encoding additional polypeptide sequences, for example, sequences that function as a marker or reporter. Examples of marker or reporter genes include -lactamase, chloramphenicol acetyl-transferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding -galactosidase), green fluorescent protein (GFP), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter. Generally, the hybrid polypeptide will include a first portion and a second portion; the first portion being a MASP-3 polypeptide and the second portion being, for example, the reporter described above or an immunoglobulin constant region.

The expression systems that may be used for purposes of the invention include, but are not limited to, microorganisms such as bacteria (for example, E coli and. B. subtills) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention (preferably containing the nucleic acid sequence of MASP-3 (SEQ ID NO:5)); insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing MASP-3 nucleotide sequences; or mammalian cell systems (for example, COS, CHO, BHK, 293, VERO, HeLa, MDCK, W138, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors may be advantageously selected depending upon the use intended for the gene product being expressed. For example, when a large quantity of such a protein is to be produced, for the generation of pharmaceutical compositions containing MASP-3 polypeptides or for raising antibodies to those polypeptides, vectors that are capable of directing the expression of high levels of fusion protein products that are readily purified may be desirable. Such vectors include, but are not limited to, the E. coli expression vector pUR278 (Ruther et al., EMBO J. 2:1791, 1983), in which the coding sequence of the insert may be ligated individually into the vector in frame with the lacZ coding region so that a fusion protein is produced; pIN vectors (Inouye and Inouye, Nucleic Acids Res. 13:3101-3109, 1985; Van Heeke and Schuster, J. Biol. Chem. 264:5503-5509, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) can be used as a vector to express foreign genes. The virus grows in Spodoptera frugiperda cells. The coding sequence of the insert may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (for example, see Smith et al., J. Virol. 46:584, 1983; Smith, U.S. Pat. No. 4,215,051).

In mammalian host cells, a number of viral-based expression systems may be utilized. In cases where an adenovirus is used as an expression vector, the nucleic acid molecule of the invention may be ligated to an adenovirus transcription/translation control complex, for example, the late promoter and tripartite leader sequence. This chimeric gene may then be inserted in the adenovirus genome by in vitro or in vivo recombination. Insertion in a non- essential region of the viral genome (for example, region E1 or E3) will result in a recombinant virus that is viable and capable of expressing a MASP-3 gene product in infected hosts (for example, see Logan and Shenk, Proc. Natl. Acad. Sci. USA 81:3655-3659, 1984). Specific initiation signals may also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544, 1987).

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. The mammalian cell types listed above are among those that could serve as suitable host cells.

For long-term, high-yield production of recombinant proteins, stable expression is preferred. For example, cell lines which stably express the MASP-3 sequences described above may be engineered. Rather than using expression vectors which contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express MASP-3. Such engineered cell lines may be particularly useful in screening and evaluation of compounds that affect the endogenous activity of the gene product and for production of MASP-3 for theraputic uses. These methods may also be used to modify cells that are introduced into a host organism either for experimental or theraputic purposes. The introduced cells may be transient or permanent within the host organism.

A number of selection systems can be used. For example, the herpes simplex virus thymidine kinase (Wigler, et al., Cell 11:223,1977), hypoxanthine-guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., Proc. Natl. Acad. Sci. USA 77:3567, 1980; O'Hare et al., Proc. Natl. Acad. Sci. USA 78:1527, 1981); gpt, which confers resistance to mycophenolic acid (Mulligan and Berg, Proc. Natl. Acad. Sci. USA 78:2072, 1981); neo, which confers resistance to the aminoglycoside G-418 (Colberre-Garapin et al., J. Mol. Biol. 150:1,1981); and hygro, which confers resistance to hygromycin (Santerre et al., Gene 30:147, 1984).

Alternatively, any fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

MASP-3Polypeptides

The MASP-3 polypeptides described herein are those encoded by any of the nucleic acid molecules described above and include MASP-3 fragments, mutants, truncated forms, and fusion proteins. These polypeptides can be prepared for a variety of uses, including but not limited to the generation of antibodies, as reagents in diagnostic assays, for the identification of other cellular gene products or compounds that can modulate the MBLectin response, and as pharmaceutical reagents useful for the treatment of inflammation and certain disorders (described below) that are associated with activity of of the MBLectin pathway. Preferred polypeptides are substantially pure MASP-3 polypeptides, including those that correspond to the polypeptide with an intact signal sequence, the mature form of the polypeptide of the human MASP-3 polypeptide as well as polypeptides representing a part of the MASP-3 polypeptide. Especially preferred are polypeptides that are soluble under normal physiological conditions.

In particular the invention relates to polypeptides comprising an amino acid sequence identified as SEQ ID NO 5 or a functional equivalent of SEQ ID NO 5, and/or an amino acid sequence identified as SEQ ID NO 1 or a functional equivalent of SEQ ID NO 1, and/or an amino acid sequence identified as SEQ ID NO 2 or a functional equivalent of SEQ ID NO 2, and/or an amino acid sequence identified as SEQ ID NO 3 or a functional equivalent of SEQ ID NO 3.

In one embodiment the polypeptide may be defined as a polypeptide having a molecular mass of about 110 kDa under non-reducing conditions on an SDS-PAGE, such as said polypeptide containing the sequence identified as SEQ ID NO 5.

In another embodiment the polypeptide may be defined as a polypeptide having a molecular mass of about 48 kDa under reducing conditions on an $DS-PAGE, such as a polypeptide containing the sequence identified as SEQ ID NO 5.

The invention also encompasses polypeptides that are functionally equivalent to MASP-3. These polypeptides are equivalent to MASP-3 in that they are capable of carrying out one or more of the functions of MASP-3 in a biological system. Preferred MASP-3 polypeptides have 20%, 40%, 50%, 75%, 80%, or even 90% of the activity of the full-length, mature human form of MASP-3. Such comparisons are generally based on an assay of biological activity in which equal concentrations of the polypeptides are used and compared. The comparison can also be based on the amount of the polypeptide required to reach 50% of the maximal activity obtainable.

Functionally equivalent proteins can be those, for example, that contain additional or substituted amino acid residues. Substitutions may be made on the basis of similarity in polarity, charge, solubility, hydrophobicity, hydrophilicity, and/or the amphipathic nature of the residues involved. Amino acids that are typically considered to provide a conservative substitution for one another are specified in the summary of the invention. D-amino acids may be introduced in order to modify the half-life of the polypeptide.

Polypeptides that are functionally equivalent to MASP-3 (e.g. SEQ ID NO:5) can be made using random mutagenesis techniques well known to those skilled in the art (and the resulting mutant MASP-3 proteins can be tested for activity). It is more likely, however, that such polypeptides will be generated by site-directed mutagenesis (again using techniques well known to those skilled in the art). These polypeptides may have an increased function, i.e., a greater ability to activate the MBLectin pathway. Such polypeptides can be used to enhance the activity of MBLectin pathway immune function.

To design functionally equivalent polypeptides, it is useful to distinguish between conserved positions and variable positions. This can be done by aligning the sequence of MASP-3 cDNAs that were obtained from various organisms. Skilled artisans will recognize that conserved amino acid residues are more likely to be necessary for preservation of function. Thus, it is preferable that conserved residues are not altered. Such conserved residues could be the three amino acids forming the so-called catalytic triad (His-497, ASP-553, Ser-664, of SEQ ID NO 5.) in the serine protease domain.

Mutations within the MASP-3 coding sequence can be made to generate MASP-3 peptides that are better suited for expression in a selected host cell. Introduction of a glycosylation sequence can also be used to generate a MASP-3 polypeptide with altered biological characteristics.

The invention also features methods for assay of polymorphisms within the polypeptide sequence comprising MASP-3 or its precursor. This may be accomplished by a number of techniques. For example, the purified polypeptide is subjected to tryptic digestion and the resulting fragments analyzed by either one-or two dimensional electrophoresis. The results from analysis of a sample polypeptide are compared to the results using a known sequence. Also the analysis may encompass separation of a biological sample (e.g., serum or other body fluids) by either one- or two-dimensional electrophoresis followed by transfer of the separated proteins onto a membrane (western blot). The membrane is then reacted with antibodies against MASP-3, followed by a secondary labelled antibody. The staining pattern is compared with that obtained using a sample with a known sequence or modification.

The polypeptides of the invention can be expressed fused to another polypeptide, for example, a marker polypeptide or fusion partner. For example, the polypeptide can be fused to a hexa-histidine tag to facilitate purification of bacterially expressed protein or a hemagglutinin tag to facilitate purification of protein expressed in eukaryotic cells. The MASP-3 polypeptide of the invention, or a portion thereof, can also be altered so that it has a longer circulating half-life by fusion to an immunoglobulin Fc domain (Capon et al., Nature 337:525-531, 1989). Similarly, a dimeric form of the MASP-3 polypeptide can be produced, which has increased stability in vivo.

In order to use the polypeptide for diagnostic purposes the polypeptide may be conjugated to a label or toxin.

Thus, the invention further provides detectably labeled, immobilized and toxin conjugated forms of the peptides, antibodies and fragments thereof. The antibodies may be labeled using radiolabels, fluorescent labels, enzyme labels, free radical labels, avidin-biotin labels, or bacteriophage labels, using techniques known to the art (Chard, Laboratory Techniques in Biology, “An Introduction to Radioimmunoassay and Related Techniques,” North Holland Publishing Company (1978).

Typical fluorescent labels include fluorescein isothiocyanate, rhodamine, phycoerythrin, phycocyanin, allophycocyanin, and fluorescamine.

Typical chemiluminescent compounds include luminol, isoluminol, aromatic acridinium esters, imidazoles, and the oxalate esters.

Typical bioluminescent compounds include luciferin, and luciferase. Typical enzymes include alkaline phosphatase, B-galactosidase, glucose-6-phosphate dehydrogenase, maleate dehydrogenase, glucose oxidase, and peroxidase.

The polypeptides of the invention can be chemically synthesized (for example, see Creighton, “Proteins: Structures and Molecular Principles,” W. H. Freeman & Co., NY, 1983), or, perhaps more advantageously, produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984).

The invention also features polypeptides that interact with MASP-3 (and the genes that encode them) and thereby alter the function of MASP-3 interacting polypeptides can be identified using methods known to those skilled in the art. One suitable method is the “two-hybrid system,” which detects protein interactions in vivo (Chien et al., Proc. Natl. Acad. Sci. USA, 88:9578, 1991). A kit for practicing this method is available from Clontech (Palo Alto, Calif.).

Anti-MASP-3 Antibodies

Human MASP-3 polypeptides (or immunogenic fragments or analogs) can be used to raise antibodies useful in the invention; such polypeptides can be produced by recombinant techniques or synthesized (see, for example, “Solid Phase Peptide Synthesis,” supra; Ausubel et al., supra). In general, the peptides can be coupled to a carrier protein, such as KLH, as described in Ausubel et al., supra, mixed with an adjuvant, and injected into a host mammal. Also the carrier could be PPD. Antibodies can be purified by peptide antigen affinity chromatography.

In particular, various host animals can be immunized by injection with a MASP-3 protein or polypeptide. Host animals include rabbits, mice, guinea pigs, rats, and chickens. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Potentially useful human adjuvants include BCG (bacille Calmette-Guerin) and Corynebacterium parvum. Immunizations may also be carried out by the injection of DNA encoding MASP-3 or parts thereoff. Polyclonal antibodies are heterogeneous populations of antibody molecules that are contained in the sera of the immunized animals.

The invention preferably relates to an antibody produced by administering an MASP-3 polypeptide, or part of the MASP-3 polypeptide, or DNA encoding any such polypeptide, according to claim 1 to an animal with the aim of producing antibody. It is preferred that said antibody selectively binds to MASP-3.

Antibodies within the invention therefore include polyclonal antibodies and, in addition, monoclonal antibodies, humanized or chimeric antibodies, single chain antibodies, Fab fragments, F(ab′)₂ fragments, and molecules produced using a Fab expression library, and antibodies or fragments produced by phage display techniques.

Monoclonal antibodies, which are homogeneous populations of antibodies to a particular antigen, can be prepared using the MASP-3 proteins described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, N.Y., 1981; Ausubel et al., supra).

In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. (In the case of chckens, the immunoglobulin class can also be IgY.) The hybridoma producing the mAb of this invention may be cultivated in vitro or in vivo. The ability to produce high titers of mAbs in vivo makes this the presently preferred method of production, but in some cases, in vitro production will be preferred to avoid introducing cancer cells into live animals, for example, in cases where the presence of normal immunoglobulins coming from the acitis fluids are unwanted, or in cases involving ethical considerations.

Once produced, polyclonal, monoclonal, or phage-derived antibodies are tested for specific MASP-3 recognition by Western blot or immuno-precipitation analysis by standard methods, e.g., as described in Ausubel et al., supra. Antibodies that specifically recognize and bind to MASP-3 are useful in the invention. For example, such antibodies can be used in an immunoassay to monitor the level of MASP-3 produced by an animal (for example, to determine the amount or subcellular location of MASP-3).

Preferably, antibodies of the invention are produced using fragments of the MASP-3 protein which lie outside highly conserved regions and appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

In some cases it may be desirable to minimize the potential problems of low affinity or specificity of antisera. In such circumstances, two or three fusions can be generated for each protein, and each fusion can be injected into at least two rabbits. Antisera can be raised by injections in a series, preferably including at least three booster injections.

Antisera is also checked for its ability to immunoprecipitate recombinant MASP-3 proteins or control proteins, such as glucocorticoid receptor, CAT, or luciferase.

The antibodies can be used, for example, in the detection of the MASP-3 in a biological sample as part of a diagnostic assay. Antibodies also can be used in a screening assay to measure the effect of a candidate compound on expression or localization of MASP-3. Thus, the antibody may be coupled to a compound comprising a detectable marker for diagnostic purposes. Such maker or label being as described above. Additionally, such antibodies can be used in conjunction with the gene therapy techniques described to, for example, evaluate the normal and/or engineered MASP-3-expressing cells prior to their introduction into the patient. Such antibodies additionally can be used in a method for inhibiting abnormal MASP-3 activity.

In addition, techniques developed for the production of “chimeric antibodies” (Morrison et al., Proc. Natl. Acad. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778, 4,946,778, and 4,704,692) can be adapted to produce single chain antibodies against a MASP-2 protein or polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Antibodies to MASP-3 can, in turn, be used to generate anti-idiotype antibodies that resemble a portion of MASP-3 using techniques well known to those skilled in the art (see, e.g., Greenspan et al., FASEB J. 7:437,1993; Nissinoff, J. Immunol. 147:2429, 1991). For example, antibodies that bind to MASP-3 and competitively inhibit the binding of a ligand of MASP-3 can be used to generate anti-idiotypes that resemble a ligand binding domain of MASP-3 and, therefore, bind and neutralize a ligand of MASP-3 such as MBL. Such neutralizing anti-idiotypic antibodies or Fab fragments of such anti-idiotypic antibodies can be used in therapeutic regimens.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

The methods described herein in which anti-MASP-3 antibodies are employed may be performed, for example, by utilizing pre-packaged diagnostic kits comprising at least one specific MASP-3 nucleotide sequence or antibody reagent described herein, which may be conveniently used, for example, in clinical settings, to diagnose patients exhibiting symptoms of the disorders described below.

Quantitative Assays of MASP-3

As an example only, quantitative assays may be devised for the estimation of MASP-3 concentrations in body fluids or organ (biopsy) extracts. Such assays may be fluid phase or solid phase. Examples are competitive and non-competitive ELISAs. As an example of the latter, microtiter wells are coated with anti-MASP-3 antibody, incubated with samples, and the presence of MASP-3 visualized with enzyme-labelled antibody followed by substrate that is cleaved into a colored compound. Alternatively, a label such as europium may be used and the detection made by use of time resolved fluorometry.

Assays for MASP-3 Antigen.

MASP-3 protein is conveniently estimated as antigen using one of the standard immunological procedures. Thus, the invention relates to a method for detecting mannan-binding lectin associated serine protease-3 (MASP-3) in a biological sample, said method comprising:

(a) obtaining a biological sample;

(b) contacting said biological sample with a MASP-3 polypeptide specific binding partner that specifically binds MASP-3; and

(c) detecting said complexes, if any, as an indication of the presence of mannan-binding fectin associated serine protease-3 in said sample.

The binding partner may be any molecule capable of selectively binding to MASP-3 and capable of being detectable, such as by labelling with a detectable label. The specific binding partner may thus be an antibody as described herein, or a mannan-binding lectin (MBL), in particular a MBL/MASP-2 complex.

As an example only, a quantitative TRIFMA (time resolved immunofluorometric assay) for MASP-3 was constructed by 1) coating microtitre wells with 1 g anti-MASP-3 antibody; 2) blocking with Tween-20; 3) applying test samples, e.g. diluted plasma or serum samples: 4) applying Eu-labelled anti-MASP-3 antibody; 5) applying enhancement solution (Wallac Ltd): 6) reading the Eu on a time resolved fluorometer. (Estimation by ELISA may be carried out similarly, e.g. by using biotin-labelled anti-MASP-3 in step 4; alkaline phosphatase-labelled avidin in step 5; 6) apply substrate; and 7) read the colour intensity.) Between each step, the plate was incubated at room temperature and washed, except between step 6 and 7. A calibration curve may be constructed using dilutions of pooled normal plasma, arbitrarily said to contain 1 unit of MASP-3 per ml.

Assays may be similarly constructed using antibodies, polyclonal or monoclonal or recombinant antibodies, which reacts with MASP-3, natural or recombinant, or parts thereof.

Through the use of antibodies reacting selectively with intact MASP-3 or with activation products, or through combination of antibodies against various parts of the molecule, assays may be constructed for the estimation of the activation in vivo of the MBLectin pathway. These assays will be useful for the determination of inflammation caused by the activation of this pathway.

Assays of the functional activity of MASP-3, either alone or as part of the MBL/MASP complex may be performed by several methods. The activity of MASP-3 to inhibit the C4 cleaving effect of MBL/MASP-2 complex may be assayed by the following method for detecting MASP-3, said method comprising an assay for MASP-3 activity, comprising the steps of

applying a sample comprising a predetermined amount of MBL/MASP-2 complexes to a solid phase obtaining bound complexes,

applying a predetermined amount of MASP-3 to the bound complexes

applying at least one complement factor to the complexes,

detecting the amount of cleaved complement factors,

correlating the amount of cleaved complement factors to the amount of MASP-3, and

determining the activity of MASP-3.

This assay may be carried out for various concentrations of MASP-3 to obtain a calibration curve.

To use the assay as a functional assay of MASP-3 in a sample, such as a serum sample, the method comprises the steps:

applying a sample comprising a predetermined amount of MBL/MASP-2 complexes to a solid phase obtaining bound complexes,

applying a sample to the bound complexes

applying at least one complement factor to the complexes,

detecting the amount of cleaved complement factors,

correlating the amount of cleaved complement factors to the activity of MASP-3 in the sample.

Whereby the correlation is conducted in relation to a standard calibration curve as the one described above.

The solid phase may be any coating capable of binding MBL, such as a mannan coating.

The complement factor preferably used in the present method is a complement factor cleavable by the MBL/MASP-2 complex, such as C4. However, the complement factor may also be selected from C3 and C5.

The cleaved complement factor may be detected by a variety of means, such as by of antibodies directed to the cleaved complement factor.

In the following an example of a test for the activity of MASP-3 is given, wherein, the test sample is applied onto mannan-coated micro wells and C4 is added to estimate the C4-cleaving activity, or C3 is added to estimate the C3 cleaving activity of the generated C3 convertase. Assay of MASP-3 not occurring as part of the MBL/MASP complex is carried out similarly, but MBL is added either to the micro well or to the sample before adding this to the mannan-coated well. Before the addition of MBL/MASP-2 complex the sample may be depleted of MBL and MBL/MASP-1 and MBL/MASP-2 and MBL/MASP-3 complexes by treatment with solid phase mannan, e.g. attached to beads, or by solid phase anti-MBL antibodies, or by treatment with a suitable concentration of a precipitating agent, e.g., PEG, which precipitates the complex but leaves MASP-3 in the supernatant. The assay is carried out at conditions which minimize or eliminate interference from the classical complement activation pathway and the alternative complement activation pathway.

Activation of the classical complement pathway is preferably inhibited to reduce the artifacts of the assay. It is preferred that the inhibition is conducted by carrying out the assay at high ionic strength, such as wherein the salt concentration is in the range of from 0.3 M to 10 M, such as from 0.5 M to 5 M, such as from 0.7 M to 2 M, such as from 0.9 M to 2 M, such as about 1.0 M. The salts used may be any one or more salts suitable for the assay, such as salts selected from NaCl, KCl, MgCl₂, CaCl₂, NaI, KCl, MgI₂, CaI₂, from NaBr, KBr, MgBr₂, CaBr₂, Na₂S₂O₃, (NH₄)₂SO₄, and NH₄HCO₃.

The inhibition of the alternative pathway may be carried out by diluting the sample at least 5 times, such as at least 10 times, such as at least 20 times or more, before conducting the assay.

Assays for MASP-3 activity of the MBL/MASP complex.

MASP-3 may be estimated by its capacity to activate or inactivate the complement system. When C4 is cleaved by MBL/MASP an active thiol ester is exposed and C4 becomes covalently attached to nearby nucleophilic groups. A substantial part of the C4b will thus become attached to the coated plastic well and may be detected by anti-C4 antibody. A quantitative TRIFMA for MASP-3 activity was constructed by 1) coating microtitre wells with 1 g mannan in 100 l buffer; 2) blocking with Tween-20; 3) applying MBL/MASP-2 complexes at a predetermined amount, applying test samples, e.g. diluted plasma or serum samples: 5) applying purified complement factor C4 at 5 g/ml; 6) incubate for one hour at 37° C.; 7) applying Eu-labelled anti-C4 antibody; 8) applying enhancement solution; and 9) reading the Eu by time resolved fluorometry. (Estimation by ELISA may be carried out similarly, e.g. by applying biotin-labelled anti-C4 in step 7; 8) apply alkaline phosphatase-labelled avidin; 9) apply substrate; and 10) read the colour intensity). Between each step the plate was incubated at room temperature and washed, except between step 8 and 9. A calibration curve can be constructed using dilutions of one selected normal plasma, arbitrarily said to contain 1 unit of MASP-3 activity per ml. The assay is preferably carried out at conditions which preclude activation of C4 by the classical or alternative complement activation pathways. The activation of C4 was completely inhibited by the serine protease inhibitor benzamidine. Activation of the classical pathway is effectively eliminated by carrying out step 3) in the presence of sufficiently high ionic strength (0.7 to 2.0 MNaCl; preferably about 1.0 M NaCl) which does not interfere with the MBL/MASP complex but comletely destroys the C1qrs complex; activation of the alternative pathway is effectively precluded by assaying at dilution as described above.

The amount of C4b being less when the assay is conducted in the presence of MASP-3 than in the absence of MASP-3, indicating that MASP-3 is an inhibitor of complement activation of MBL/MASP-2 complex.

Assays for the estimation of free MASP-3 activity.

The estimation of MASP-3 activity in samples from MBL-deficient individuals is carried out on wells coated with MBL/MASP-2 complexes. The estimation of free MASP-3 in samples from individuals with MBL is carried out by first removing MBL/MASP-1 and MBL/MASP-2 and MBL/MASP-3 complexes by incubating with Sepharose-coupled mannan (300 l of 10 fold diluted plasma or serum is incubated with 10 l beads), and then analyzing the supernatant. The assay may be carried out as described above, or as the following assay:

The assay carried out in the TRIFMA formate proceeds as follows: 1) coating microtitre wells with 1 g mannan in 100 l buffer; 2) blocking with Tween-20; 3) incubate sample at a 1000 fold dilution in buffer with 100 ng of MASP-free MBL/ml, and applying 100 l of the mixture per well; 4) incubate over night at 4° C.; 4) wash and applying purified complement factor C4 at 5 g/ml; 5) incubate for one hour at 37° C.; 6) applying Eu-labelled anti-C4 antibody; 7) applying enhancement solution; and 8) reading the Eu by time resolved fluorometry. (Estimation by ELISA may be carried out similarly, e.g. by applying biotin-labelled anti-C4 in step 6; 7) apply alkaline phosphatase-labelled avidin; 8) apply substrate; and 9) read the colour intensity.) Between each step the plate was washed, except between step 7 and 8. A calibration curve may be constructed using dilutions of one selected MBL-deficient plasma, arbitrarily said to contain 1 unit of MASP-3 activity per ml. The assay is carried out at conditions which preclude activation of C4 by the classical or alternative complement activation pathways (see above).

Assays estimating the activity of MASP-3 or quantity of MASP-3 may be used for diagnostic and treatment purposes in samples from individuals, notably those suffering from infectious or inflammatory diseases.

MASP-3 Functionality

It is important to realise that only a minor proportion of these proteases are associated with MBL in serum, as has been demonstrated for MASP-1 and MASP-2^{18,19}. By depleting serum of MBL complexes and analysing for residual MASP-3, the same was found the same to be true for this protein.

MASP-3 is believed to exert an inhibitory effect on the complement activation, particular when bound to MBL/MASP-2 complexes.

Due to the fact that only a minor proportion of MASP-3 is bound to MBL in serum, it is further believed that MASP-3 also exerts a stimulating effect on for example the complement activation, either directly or bound to other protein, such as by forming a MBL/MASP-3 complex.

MASP-3 for Therapy

Therapeutic use of components specified in the claims may be applied in situations where a constitutional or temporary deficiency in MASP-3 renders the individual susceptible to one or more infections, or situations where the individual cannot neutralize an established infection. MASP-3 or MBL/MASP complexes can be administered, preferably by intravenous infusions, in order to improve the individual's immune defense.

Without being bound by theory, it is believed that MASP-3 is required for the powerful antimicrobial activity of the MBL/MASP complex, and deficiency in MASP-3, either genetically determined or acquired, will therefore compromise an individual's resistance to infections and ability to combat established infections. Reconstitution with natural or recombinant MASP-3 is a useful treatment modality in such situations. Recombinant MASP-3 may be in the form of the whole molecule, parts of the molecule, or the whole or part thereof attached by any means to another structure in order to modulate the activity. The recombinant products may be identical in structure to the natural molecule or slightly modified to yield enhanced activity or decreased activity when such is desired.

Stimulation

MASP-3 may in one embodiment have a stimulating effect on the complement activation, such as by direct activation of the complement system or through binding to MBL.

Reconstitution therapy with MBL, either natural or recombinant, requires that the recipient has sufficient MASP-3 for the expression of MBL/MASP activity. Thus, MASP-3 must be included in the therapeutic preparation when the patient has insufficient MASP-3 activity.

Administration of functional MASP-3 or MBL/MASP-3 complexes or any functional derivative or variant thereof by e.g. intravenous infusions in order to improve the individual's immune defense represents one preferred method of treatment by therapy in accordance with the present invention. However, other methods of treatment may comprise curative treatment and/or prophylaxis of e.g. the immune system and reproductive system by humans and by animals.

Conditions to be treated are not limited to presently known conditions for which there exist a need for treatment. The condition comprise generally any condition in connection with current and/or expected need or in connection with an improvement of a normal condition. In particular, the treatment is a treatment of a condition of deficiency of MBL. In another aspect of the present invention the manufacture is provided of a medicament comprising a pharmaceutical composition comprising functional MASP-3 or MBL/MASP complexes, or any variant thereof, intended for treatment of conditions comprising cure and/or prophylaxis of conditions of diseases and disorders of e.g. the immune system and reproductive system by humans and by animals having said functional units acting like those in humans.

Said diseases, disorders and/or conditions In need of treatment with the compounds of the invention comprise e.g. treatment of conditions of deficiency of MBL, treatment of cancer and of infections in connection with immunosuppressive chemotherapy including in particular those infections which are seen in connection with conditions during cancer treatment or in connection with implantation and/or transplantation of organs. The invention also comprises treatment of conditions in connection with recurrent miscarriage.

Thus, in particular the pharmaceutical composition comprising MASP-3 or a functional variant thereof may be used for the treatment and/or prevention of clinical conditions selected from infections, MBL deficiency, cancer, disorders associated with chemotherapy, such as infections, diseases associated with human immunodeficiency virus (HIV), diseases related with congenital or acquired immunodeficiency. More particularly, chronic inflammatory demyelinating polyneuropathy (CIDP, Multi-focal motoric neuropathy, Multiple scelrosis, Myasthenia Gravis, Eaton-Lambert's syndrome, Opticus Neuritis, Epilepsy; Primary antiphosholipid syndrome; Rheumatoid arthritis, Systemic Lupus erythematosus, Systemic scleroderma, Vasculitis, Wegner's granulomatosis, Sjøgren's syndrome, Juvenile rheumatiod arthritis; Autoimmune neutropenia, Autoimmune haemolytic anaemia, Neutropenia; Crohn's disease, Colitis ulcerous, Coeliac disease; Asthma, Septic shock syndrome, Chronic fatigue syndrome, Psoriasis, Toxic shock syndrome, Diabetes, Sinuitis, Dilated cardiomyopathy, Endocarditis, Atherosclerosis, Primary hypo/agammaglobulinaemia including common variable immunodeficiency, Wiskot-Aldrich syndrome and serve combined immunodefiency (SCID), Secondary hypo/agammaglobulinaemia in patients with chronic lymphatic leukaemia (CLL) and multiple myeloma, Acute and chronic idiopathic thrombocytopenic purpura (ITP), Allogenic bone marrow transplantation (BTM), Kawasaki's disease, and Guillan-Barre's syndrome.

In particular the MASP-3 composition may be administered to prevent and/or treat infections in patients having clinical symptoms associated with congenital or acquired MBL deficiency or being at risk of developing such symptoms. A wide variety of conditions may lead to increased susceptibility to infections in MBL-deficient individuals, such as chemotherapy or other therapeutic cell toxic treatments, cancer, AIDS, genetic disposition, chronic infections, and neutropenia.

The infection may be caused by any pathogenic or parasitic agent including any bacterial agent and any viral agent. The treatment may be directed against a local infection, such as e.g. a meningeal infection, or the treatment may be aimed at combatting an acute systemic infection that may develop into a life threatening infection unless treated. The inflammatory condition may also result from an autoimmune condition.

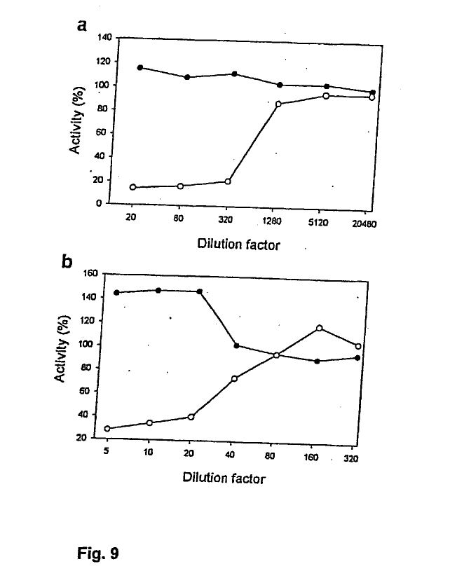

In another embodiment MASP-3 has an inhibitory effect on complement activation, in particular activation of C4. An examination of the biological activity of MASP-3 carried out by using recombinant proteins produced in a mammalian expression system revealed a pronounced inhibitory activity of rMASP-3 on the activation of C4 by natural MBL complexes (FIG. 9a). The activity of rMBL-rMASP-2 complexes was also inhibited by rMASP-3 (FIG. 9b).

There is accordingly provided a method for inhibiting complement activation by inhibiting the MBL pathway, said method comprising the step of administering an effective amount of MASP-3, or a functional variant thereof, to an individual in need of complement down-regulation and/or complement inhibition.

In one preferred embodiment of the present invention there is provided a method for inhibiting the activation of C4 complement by inhibiting the MBL pathway, said method comprising the step of administering an effective amount of MASP-3 or a functional variant thereof to an individual in need of C4 down-regulation and/or C4 inhibition.

There is also provided a method for inhibiting MASP-2 activity, said method comprising the step of administering an effective amount of MASP-3, or a functional variant thereof, to an individual in need of MASP-2 down-regulation and/or MASP-2 inhibition. In one presently preferred embodiment MASP-3 is capable of inhibiting MASP-2 complexes with MBL.

Thus, there is provided a method for inhibiting or treating an inflammatory condition in an individual, in particular a condition related to complement activation through MBL/MASP complexes, said method comprising the step of administering an effective amount of MASP-3, or a functional variant thereof, to an individual in need of treatment for an inflammation. The inflammatory condition may be chronic, such as e.g. rheumatoid arthritis or systemic lupus erythematosus, or the inflammatory condition may be an acute inflammatory condition. The treatment according to the invention is in one such embodiment directed against treatment of reoxygenated ischemic tissues, such as the inflammatory condition may also result from an autoimmune condition after an acute nyocardial infarction or brain ischemia.

In a still further embodiment there is provided a method for treating in an individual suffering from a disorder resulting from an imbalanced cytokine network, e.g. a disorder involving or resulting from an unfavourable TNF response to bacterial lipo-polysaccharides, said method comprising the step of administering an effective amount of MASP-3, or a functional variant thereof, to an individual in need thereof.

The route of administration may be any suitable route, such as intravenously, intramusculary, subcutanously or intradermally. Also, pulmonal or topical administration is envisaged by the present invention.

Use of MASP-3 for Clinical Purposes

The polypeptide according to the invention may be used for a variety of clinical purposes, such as for administration as a pharmaceutical composition. Thus, in one aspect the present invention relates to the use of the polypeptide according to the invention, or a compound as defined herein for preparation of a pharmaceutical composition.

The pharmaceutical composition is preferably capable of being administered parenterally, such as intramusculary, intravenously, or subcutaneously, or capable of being administered orally.

As discussed above with respect to therapy with MASP-3 the pharmaceutical composition may be used for a wide variety of diseases and condition, such as the treatment of MASP-3 deficiency, or for the inhibition of the MBL/MASP complexes.

Assays for MASP-3

Therapy with MASP-3 (or MASP-3 inhibitors) must usually be preceded by the estimation of MASP-3 in serum or plasma from the patient. Examples of such assays are described below.

Inhibition of MASP-3 Activity.

Inhibitors of the biological activity of MASP-3 may be employed to control the complement activating activity and inflammatory activity of MASP-3 or for neutralizing the inhibitory effect of MASP-3 thus giving an overall increase of the activity of the MBL/MASP complex. Such inhibitors may be substrate analogues representing target structures for the enzymatic activity of MASP-3. Inhibitors may be of peptide nature, modified peptides, or any organic molecule which inhibits the activity of MASP-3 competitively or non-competitively. The inhibitor may be modified to stay in circulation for short or longer time, and constructed to be given by injection or perorally. Inhibitors may be fragments of MASP-3, produced from natural or recombinant MASP-3, by chemical or enzymatic procedures. Inhibitors may be naturally occurring shorter forms of MASP-3. Inhibitors may be mutated forms of MASP-3. Inhibitors may be in soluble form or coupled to a solid phase. A solid phase could be a compatible surface such as used in extracorporal blood or plasma flow devices.

The MASP-3 activity may be inhibited by a compound capable of inhibiting the complex formation of MBL and MASP-3. The compound may be any compound capable of binding to MBL/MASP-2 complex without exhibiting the MASP-3 effect. Accordingly, the compound may comprise a polypeptide as defined herein or a fragment thereof capable of binding MBL.

In another embodiment the compound may be or comprise an antibody as defined herein capable of binding MASP-3 thereby inhibiting the MASP-3 activity.

Also, such a compound may be capable of disrupting the complex formation of MBL and MASP-3 thereby inhibiting the activity of MASP-3.

Microbial carbohydrates or endogenous oligosaccharides may provoke undesirable activation of the MBL/MASP complex resulting in damaging inflammatory responses. This pathophysiological activity may be reduced though the administration of inhibitors of MASP-3 activity such as Pefabloc. Also other enzyme inhibitors (C1 Inhibitor, ₂-macroglobulin, Trasylol (Aprotenin), PMSF, benzamidine, etc.) have proved effective when assayed in the TRIFMA for MASP-3 activity. Obviously, when designing inhibitors for in vivo use toxicity is a major consideration, and highly specific inhibitors can be assumed to be less toxic than more broadly reactive inhibitors. Specific inhibitors may be generated through using peptides, peptide analogues or peptide derivatives representing the target structures. Another type of inhibitors may be based on antibodies (or fragments of antibodies) against the active site of MASP-3 or other structures on MASP-3 thus inhibiting the activity of MASP-3. Inhibitors may also be directed towards inhibition of the activation of MASP-3. Another type of inhibitor would prevent the binding of MASP-3 to MBL and thereby the activation of MASP-3. The drain fragment of MASP-3 may be a suitable inhibitor of this type. More specifically one can localize the precise part of the polypeptide chain which mediates the binding of MASP-3 to MBL and use the synthetic peptide or analogous structures as inhibitor. Inhibitors may be substituted with D amino acids for L-amino acids.

Also, inhibitors could be RNA or single stranded DNA isolated by SELEX (systemic evolution of ligands by exponential enrichment) using MASP-3 or fragments thereof as selecting molecule capable of binding to the MASP-3 molecule. Another method for inhibiting the activity of MASP-3 is by administering to the subject a compound that inhibits expression of MASP-3, such as a MASP-3 anti-sense nucleic acid sequence.

MASP-3 activity may also be controlled by control of the conversion of the proenzyme form of MASP-3 into activated MASP-3.

Pharmaceutical Composition

The pharmaceutical compositions according to the present invention may comprise one or more polypeptides or compounds according to this invention, optionally further comprising pharmaceutically acceptable carriers.

According to the methods of the invention the compositions can be administered by injection by gradual infusion over time or by any other medically acceptable mode. The administration may, for example, be intravenous, intraperitoneal, intramuscular, intracavity, subcutaneous or transdermal. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloilate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation. When the compositions of the invention are administered for the treatment of pulmonary disorders the compositions may be delivered for example by aerosol.

The compositions of the invention are administered in therapeutically effective amounts. As used herein, an “effective amount” of the polypeptide or compound of the invention is a dosage which is sufficient to conduct the desired associated complement activation or neutralization. The effective amount is sufficient to produce the desired effect of inhibiting associated cellular injury until the symptoms associated with the MBL mediated disorder are ameliorated or decreased. Preferably an effective amount of the polypeptide is an effective amount for preventing cellular injury. Generally, a therapeutically effective amount may vary with the subject's age, condition, and sex, as well as the extent of the disease in the subject and can be determined by one of skill in the art. The dosage may be adjusted by the individual physician or veterinarian in the event of any complication. A therapeutically effective amount typically will vary from about 0.01 mg/kg to about 500 mg/kg, such as typically from about 0.1 mg/kg to about 200 mg/kg, and often from about 0.2 mg/kg to about 20 mg/kg, in one or more dose administrations daily, for one or several days (depending of course of the mode of administration and the factors discussed above).

One of skill in the art can determine what an effective amount of a compound is by screening the MASP-3 concentration and associated complement activation in an in vitro assay.

The polypeptide and compound may be administered in a physiologically acceptable carrier. The term “physiologically-acceptable” refers to a non-toxic material that is compatible with the biological systems such of a tissue or organism. The physiologically acceptable carrier must be sterile for in vivo administration. The characteristics of the carrier will depend on the route of administration. The characteristics of the carrier will depend on the route of administration.

### REFERENCES

1) Law, S. K. A. & Reid, K. B. M. Complement, 2 ed. (Ed. Male, D.) 1-88 (In Focus, IRL Press, Oxford, 1996).

2) Ikeda, K., Sannoh, T., Kawasaki, N., Kawasaki, T. & Yamashina, l. Serum lectin with known structure activates complement through the classical pathway. J. Biol. Chem. 262, 7451-7454 (1987).

3) Kawasaki, T., Etoh, R. & Yamashina, I. Isolation and characterization of a mannan-binding protein from rabbit liver. Biochem. Biophys. Res. Commun. 81, 1018-1024 (1978).

4) Matsushita, M. & Fujita, T. 4) Activation of the classical complement pathway by mannose-binding protein in association with a novel C1s-like serine protease J. Exp. Med. 176, 1497-1502 (1992).

5) Ji, Y-H. et al. Activation of the C4 and C2 components of complement by a pro teinase in serum bactericidal factor, Ra reactive factor J. Immunol. 150, 571-578 (1993).

6) Turner, M. W. Mannose-binding lectin: the pluripotent molecule of the innate immune system. Immunol. Today, 17, 532-540 (1996).

7) Kawasaki, N., Kawasaki, T. & Yamashina, I. A serum lectin (mannan-binding protein) has complement-dependent bactericidal activity. J. Biochem. 106, 483-489 (1989).

8) Kuhlman, M., Joiner, K. & Ezekowitz, R. A. B. The human mannose-binding protein functions as an opsonin. J. Exp. Med. 169, 1733-1745 (1989).

9) Sumiya, M. et al. Molecular basis of opsonic defect in immunodeficient children. Lancet 337, 1569-1570 (1991).

10) Lipscombe, R. J. et al. High frequencies in African and non-African populations of independent mutations in the mannose binding protein gene. Hum. Mol. Genet 1, 709-715 (1992).

11) Madsen H. O. et al. A new frequent allele is the missing link in the structural polymorphism of the human mannan-binding protein. Immunogenetics 40, 37-44 (1994).

12) Super, M., Thiel, S., Lu, J., Levinsky, R. J. & Turner, M. W. Association of low levels of mannan-binding protein with a common defect of opsonisation. Lancet ii, 1236-1239 (1989).

13) Garred, P., Madsen, H. O., Hofmann, B. & Svejgaard, A. Increased frequency of homozygosity of abnormal mannan-binding-protein alleles in patients with suspected immunodeficiency. Lancet 346, 941-943 (1995).

14) Summerfield, J. A. et al. Mannose binding protein gene mutations associated with unusual and severe infections in adults. Lancet 345, 886-889 (1995).

15) Nielsen, S. L., Andersen, P. L., Koch, C., Jensenius, J. C. & Thiel, S. The level of the serum opsonin, mannan-binding protein in HIV-1 antibody-positive patients. Clin. Exp. Immunol. 100, 219-222 (1995).

16) Garred, P., Madsen, H. O., Balslev, U., Hofmann, B., Pedersen, C., Gerstoft, J. and Svejgaard, A. Susceptibility to HIV infection and progression of AIDS in relation to variant alleles of mannose-binding lectin. Lancet 349, 236-240 (1997).

17) Malhotra, R. Wormald, M. R., Rudd, P. M., Fischer, P. B., Dwek, R. A. and Sim, R. B. Glycosylation changes of IgG associated with rheumatoid arthritis can activate complement via the mannose-binding protein. Nature Med. 1, 237-243 (1995).

18) Kilpatrick, D.C., Bevan, B. H. and Liston, W. A. Association between mannan-binding protein deficiency and recurrent miscarriage. Mol. Hum. Reprod. 1, 2501-2505 (1995).

19) Davies, E. J., Snowden, N., Hillarby, M. C., Carthy, D. Grennan, D. M., Thomson, W. and Ollier, W. E. R. Mannose-binding protein gene polymorphism in systemic lupus erythematosus. Arthritis Rheum. 38, 110-114 (1995).

20) Valdimarsson H, Stefansson M, Vikingsdottir T, Arason G J, Koc C, Thiel S, Jensenius J C. Reconstitution of opsonizing activity by infusion of mannan-binding lectin (MBL to MBL-deficient humans. Scand J Immunol. 1998 August; 48(2):116-23.

21) Garred, P., Madsen, H. O., Kurtzhals, J. A., et al. Diallelic polymorphism may explain variations of blood concentrations of manna n-binding protein in Eskimos but not in black Africans. Eur. J. Immunogenet. 19, 403-412 (1992).

22) Thiel, S., Vorup-Jensen, T., Stover, C. M., Schwable, W., Laursen, S. B., Poulsen, K., Willis, A. C., Eggleton, P., Hansen, S., Holmskov, U., Reid, K. B. M., Jensenius, J. C. (1997), A second serine protease associated with mannan-binding lectin that activates complement, Nature, 386, 506-510.

23) Sato, T., Endo, Y., Matsushita, M. & Fujita, T. Molecular characterization of a novel serine protease involved in activation of the complement system by mannose-binding protein. Int. Immunol. 6, 665-669 (1994).

24) Endo, Y., Sato, T., Matsushita, M. & Fujita, T. Exon structure of the gene encoding the human mannose-binding protein-associated serine protease light chain: comparison with complement C1r and C1s genes. Int Immunol. 9, 1355-1358 (1996).

25) Journat, A. & Tosi, M. Cloning and sequencing of full-length cDNA encoding the precursor of human complement component C1r. Biochem. J. 240, 783-787 (1986).

26) Lytus, S. P., Kurachi, K., Sakariassen, K. S. & Davie, E. W. Nucleotide sequence of cDNA coding for human complement C1r. Biochemistry 25, 4855-4863 (1986).

27) Mackinnon, C. M., Carter, P. E., Smyth, S. J., Dunbar, B. & Fothergill, J. E. Molecular cloning of cDNA for human complement component C1s. The complete amino acid sequence, Eur. J. Biochem. 169, 547-553 (1987).

28) Tosi, M., Duponchel, C., Meo, T. & Julier, C. Complete cDNA sequence of human complement Cls and close physical linkage of the homologous genes Cls and Clr. Biochemistry 26, 8516-8524 (1987).

29) Thiel, S., et al. Interaction of C1q and mannan-binding lectin (MBL) with C1r, C1s, MBL-associated serine protease 1 and 2 and MAp19. J. Immunol. 165, 878-887

30) Vorup-Jensen, T. et al. Distinct pathways of mannan-binding lectin (MBL)- and C1-complex autoactivation revealed by reconstitution of MBL with recombinant MBL-associated serine protease-2. J. Immunol. 165, 2093-2100.

31) Baatrup, G., Thiel, S., Isager, H., Svehag, S. E. & Jensenius, J. C. Demonstration in human plasma of a lectin activity analogous to that of bovine conglutinin. Scand. J. Immunol. 26, 355-361 (1987).

32) Volanakis, J. E. & Frank, M. M. (eds.) The Human Complement System in Health and Disease. Marcel Decker Inc., New York (1998).

33) Croix, D. A. et al. Antibody response to a dependent antigen requires B cell expression of complement receptors. J. Exp. Med. 183, 1857-1864 (1996).

34) Dempsey, P. W., Allison, M. E. D., Akkaraju, S., Goodnow, C. C. & Fearon, D. T. C3d of complement as a molecular adjuvant: Bridging innate and acquired immunity. Science 271, 348-350 (1996).

35) Summerfield, J. A., Sumiya, M., Levin, M. & Turner, M. W. Association of mutations in mannose-binding protein gene with childhood infections in consecutive hospital series. Brit Med. J. 314, 1229-1232 (1997).

36) Garred, P. et al. Association of mannose-binding lectin gene heterogeneity with severity of lung disease and survival in cystic fibrosis. J. Clin. Invest. 104, 431-437 (1999).

37) Stower, C. M. et al. Two constituents of the initiation complex of the mannan-binding lectin activation pathway of complement are encoded by a single structural gene. J. Immunol. 162, 3481-3490 (1999).

38) Takahashi, M., Endo, Y., Fujita, T. & Matsushita, M. A truncated form of man-nose-binding lectin-associated serine protease (MASP)-2 expressed by alternative polyadenylation is a component of the lectin complement pathway. Int Immunol. 11, 859-863 (1999).

39) Lu, J., Thiel, S., Wiedemann, H., Timpl, R. & Reid, K. B. M. Binding of the pentamer/hexamer forms of mannan-binding protein to zymosan activates the proenzyme C1r2C1s2 complex, of the classical pathway of complement, without involvement of C1q, J. Immunol. 144, 2287-2294 (1990).

40) Lipscombe, R. J., Sumiya, M., Summerfield, J. A. & Turner, M. W. Distinct physicochemical characteristics of human mannose binding protein expressed by individuals of differing genotypes. Immunology, 85, 660-7 (1995).

41) Matsushita, M. & Fujita, T. Cleavage of the third component of complement (C3) by mannose-binding protein-associated serine protease (MASP) with subsequent complement activation. Immunobiol. 194, 443451 (1995).

42) Terai, I., Kobayashi, K., Matsushita, M. & Fujita, T. Human serum mannose-binding lectin (MBL)-associated serine protease-1 (MASP-1): determination of levels in body fluids and identification of two forms in serum. Clin. Exp. Immunol. 110, 317-23 (1997).

43) Endo, Y. et al. Two lineages of mannose-binding lectin-associated serine protease (MASP) in vertebrates. J. Immunol. 161, 49244930 (1998).

44) Matsushita, M., Thiel, S., Jensenius, J.C., Terai, I. & Fujita, T. Proteolytic activities of two types of mannose-binding lectin-associated serine protease. J. Immunol. in press 165, 2637-2642.

45) Dodds, A. W. Small scale preparation of complement components C3 and C4. Meth. Enzymol. 223, 46-61 (1986).

### EXAMPLES

### Example 1

### Identification of MASP-3

Human plasma proteins and protein complexes, that bind to carbohydrates in a calcium-dependent manner (i.e. lectins and lectin-associated proteins), were purified by affinity chromatography on mannan- or mannose- or N-acetylglucosamine-derivatized Sepharose or TSK beads. Pooled CPD-plasma (2.5 l), diluted with buffer containing EDTA and enzyme inhibitors were passed through Sepharose 2B CL and mannan-Sepharose. A thrombin inhibitor, PPACK (D-phenylalanyl-prolyl-arginyl-chloromethyl ketone) and CaCl₂ were added. The pool was passed through Sepharose 2B-CL and mannan-Sepharose, and the proteins binding calcium-dependently to mannan-Sepharose were eluted with EDTA-containing buffer. The eluate was recalcified, passed through a GlcNAc-Sepharose column which was eluted as above to yield 20 ml “lectin preparation”.

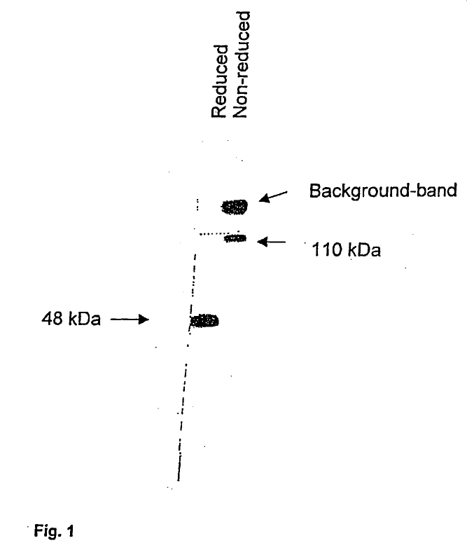

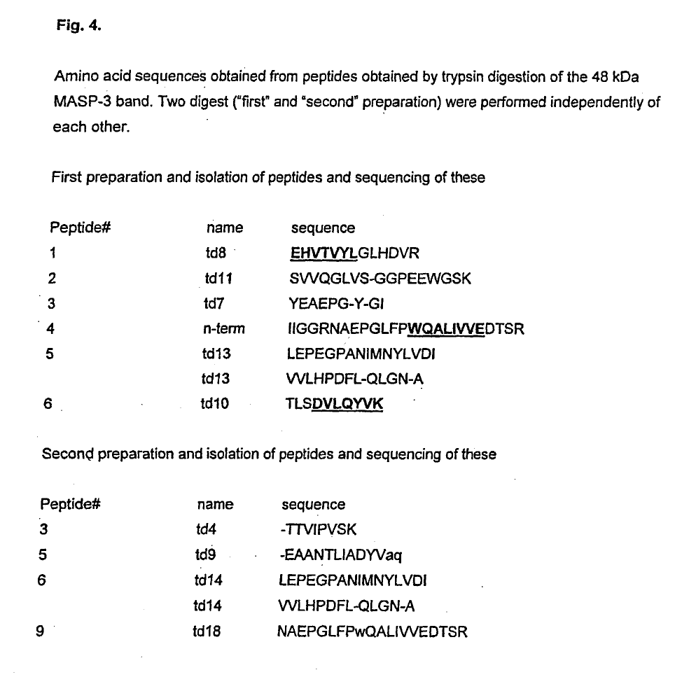

This protein preparation was analyzed by SDS-PAGE and blotting onto a PVDF-membrane. Development of the blot with chicken antibody raised against a bovine lectin preparation³¹ revealed the 52 kDa A-chain of MASP-2 as well as MBL at 32 kDa. An additional 48 kDa band was revealed by nonspecific protein staining with Coomassie Brilliant Blue. The 48 kDa band was subjected to NH₂-terminal amino acid sequence analysis. The sequence obtained (FIG. 4) showed similarity to that of the serine protease domain (the B chain) of the previously described MASPs. Antibody raised against a synthetic peptide representing the 19 NH₂-terminal amino acids (anti-pMASP-3 antiserum) recognized the 48 kDa molecule (FIG. 1, lane 1). Under non-reducing conditions a polypeptide of 110 kDa was detected using the anti-pMASP-3 antiserum (FIG. 1, lane 2), indicating the presence of intra-chain disulphide bonds.

### Example 2

### Preparation of Antibodies Against MASP-3

Animals, primed with BCG (Bacillus Calmette Guérin vaccine) were immunized with synthetic peptides coupled to PPD (tuberculin purified protein derivative). Antibody designated anti-pMASP-3 was from rabbits immunized with peptides corresponding to the first 20 amino acids (IIGGRNAEPGLFPWQALIVV) of the 48 kDa MASP-3 band. All peptides had an additional C-terminal cysteine for coupling. Monoclonal anti-MBL antibody, IgG,-kappa (clone 131-1) and control IgG₁-kappa (clone MOPC 21) were purified by Protein A affinity chromatography. For staining of Western blots antibodies were used at 1 g/ml. Bound rabbit antibodies were visualized with peroxidase-labelled goat anti-rabbit IgG followed by development using the enhanced chemiluminescence technique.

### Example 3

### MBL/MASP Complexes

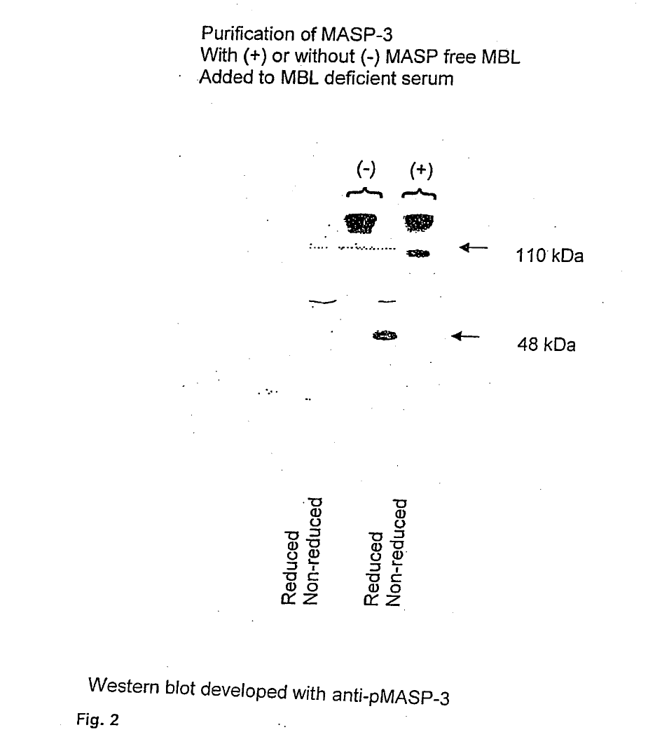

Two microgram MASP-depleted MBL was added to 1 ml MBL deficient serum and subsequently 100 microliter mannose-TSK beads were added. Also 1 ml MBL deficient serum was incubated with 100 microliter mannose-TSK beads. After incubation over night at 4 degrees celcius the beads were washed with a calcium containing buffer and subsequently an elution buffer consisting of SDS-PAGE buffer diluted 2 fold with TBS (tris buffered saline solution containing 20 mM Tris, 145 mM NaCI) containing 10 mM EDTA was added to the beads. The eluted proteins were subjected to SDS-PAGE western blotting, in both reducing and non-reducing conditions. The western blot was developed with rat anti-pMASP-3 antibody followed by HRP labelled anti-rat IgG antibody. MASP-3 was only found to be present in eluates from beads incubated with MBL-deficient serum with MASP-free MBL added and not in eluates from beads which had been incubated with MBL-deficient serum only (FIG. 2).

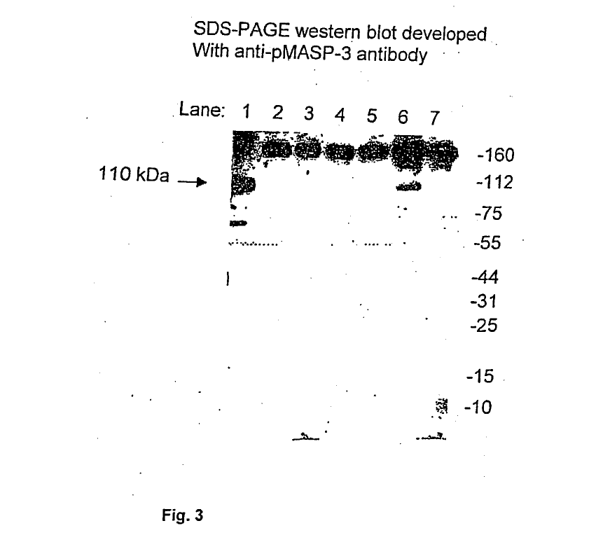

The lectin preparation (described above in example 1) was incubated in microtitre wells coated with monoclonal anti-MBL antibody, monoclonal anti-MASP-1 antibody or, as a negative control, wells coated with non-specific monoclonal immunoglobulin of the same subclass. The lectin preparation was diluted both in calcium containing buffer and in EDTA containing buffer. The proteins captured by the antibody were eluted and analyzed by SDS-PAGE/Western blotting under non-reduced conditions. The blot was developed with anti-pMASP-3 antibody. The results (FIG. 3) show that the anti-MBL antibody, in addition to binding MBL, captures MASP-3 whereas monoclonal anti-MASP-1 does not. Lane 1 represents unfractionated lectin preparation. Lanes 2-and 3 represent eluates from wells coated with non-sense IgG and incubated with lectin preparation (lane 2 in the presence of calcium, lane 3 in the presence of EDTA), while lanes 4 and 5 represent eluates from wells coated with monoclonal anti-MASP-1 antibody and incubated with lectin preparation (lane 4 in the presence of calcium, lane 5 in the presence of EDTA) and lane 6 and 7 represents eluates from wells coated with monoclonal anti-MBL antibody and incubated with lectin preparation (lane 6 in the presence of calcium, lane 7 in the presence of EDTA). The position of the 110 kDa MASP-3 band is indicated on the figure.

This experiment reveals that MASP-3 can only be found in eluates from wells coated with anti-MBL antibodies and not from wells coated with anti-MASP-1 or with non-sense. IgG. Thus MASP-3 is associated with MBL and to a much lower extent, or not at all, with MASP-1. Further it is found that the association between MBL and MASP-3 is calcium dependent.

### Example 4

### Amino Acid Sequencing of N-termini and of Peptides of the 48 kDa Polypeptide

The lectin preparation was concentrated, subjected to SDS-PAGE, and transferred to a PVDF membrane. The blot was stained with Coomassie Brilliant Blue. The band corresponding to the coomasie-stained 48 kDa band was cut out and subjected to sequencing on an Applied Biosystems protein sequencer. After production of anti-pMASP-3 antibody, a similar Western blot was performed using the anti-pMASP-3 antibody. The NH₂-termini of the protein in the 48 kDa band visualized with this antibody were sequenced and were identical to the ones obatined for the coomasie stained 48 kDa band mentioned above. Peptides were prepared by trypsin digestion of the protein in the 48 kDa band from a coomasie stained SDS-PAGE gel. The peptides were fractionated by reverse phase chromatography and the peptides in the major peaks were subjected to sequencing. The sequences obtained are given in FIG. 4.

### Example 5

### Cloning and sequencing of MASP-3

The liver is the primary site of synthesis of C1r, C1s, MASP-1 and MASP-2. Thus cDNA prepared from liver RNA was used as template for PCR with primers deduced from the obtained peptide sequences. PCR was performed on the cDNA using degenerate primers derived from the amino acid sequences WQALIVVE and EHVTVYL. The resulting PCR product was cloned into the E. coli plasmid pCRII using the TA-cloning kit (In Vitrogen) and the nucleotide sequence of the insert was determined.

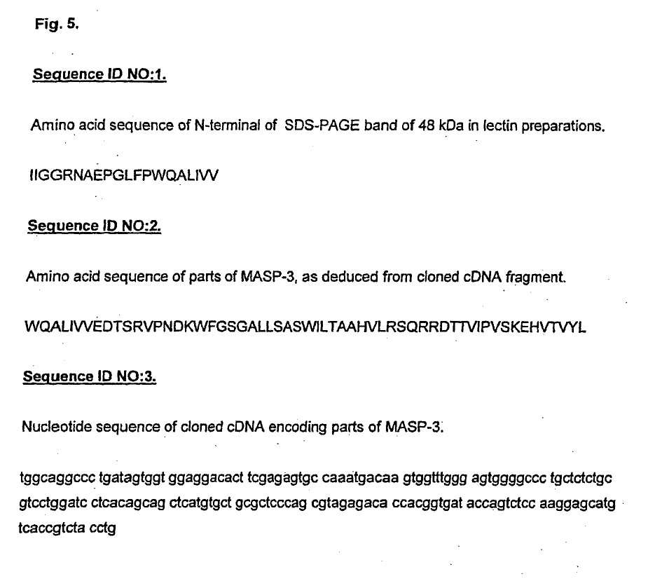

The nucleotide sequence of the resulting PCR product contained an open reading frame (ORF) with a deduced amino acid sequence confirming the sequences of the peptides from which the primers were derived as well as that of another of the sequenced peptides. The nucleotide sequence of the cDNA is shown in FIG. 5 together with the translated amino acid sequence.

### Example 6

### Comparison of MASP-3 to MASP-1, MASP-Z C1r and C1s

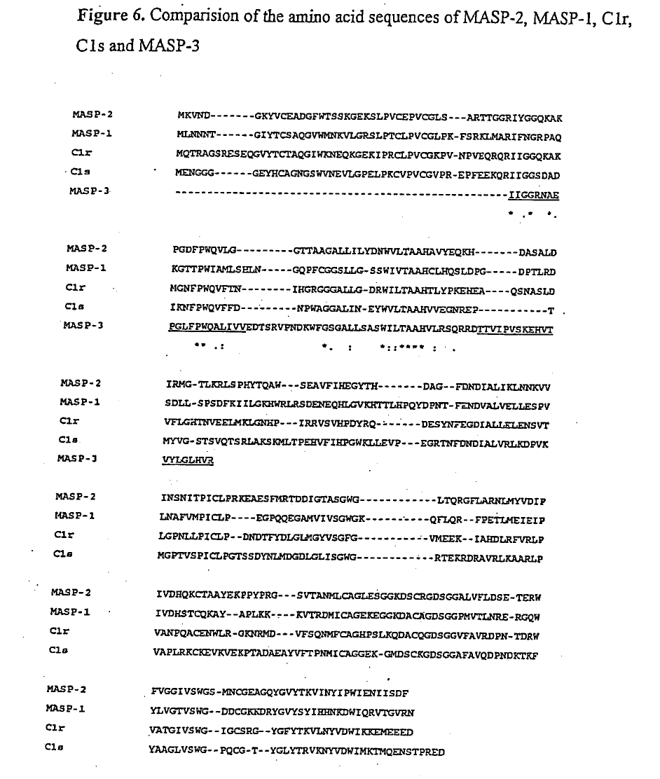

The amino acid sequence deduced from the cDNA sequence in FIG. 5 is homologous to those of MASP-1, MASP-2, C1r and C1s (FIG. 6). MASP-1, MASP-2, C1r, and C1s are all activated by cleavage of the peptide bond between the residues Arg and lie located between the second CCP domain and the serine protease domain. The resulting polypeptide chains (the largest referred to as the A chain and the smallest as the B chain) are held together by a disulphide bond. By analogy, our results indicate that the 48 kDa polypeptide, recognized by the anti-pMASP-3 antibody after SDS-PAGE under reducing conditions, is part of the B chain of MASP-3. Identities and similarities between the four proteins were studied based on the alignment in FIG. 6. Identical residues in all four species are indicated by asterisks. The potential cleavage site between Arg and lle residues, which generates A and B chains, is identical to the site where the serine protease domain of MASP-3 starts. The sequences obtained by amino acid sequencing of peptides of the 48 kDa band are underlined. Only the MASP-1 sequence contains the histidine loop, characteristic of trypsin-like serine proteases^{23,24}.

### Example 7

### MASP-3 and the Initiator Complexes of the MBL Complement Activation Pathway

The complement system represents an antimicrobial defence mechanism of major clinical importance³², with a well-established role in the adaptive immune response^{33,34}. A surprising development has been the recent discovery of a mannan-binding lectin (MBL) pathway^{2,4,5,22} of complement activation. Accumulating clinical evidence demonstrates the importance of human MBL in non-adaptive defence against invading microorganisms^{2,3,5,38}, but the molecular characteristics and mechanisms of the initiating complex remain obscure. Two serine proteases, MASP-1 and MASP-2^{4,5,22} and a peptide, MAp19³⁷ or sMAP³⁸, have been reported to be associated with MBL, the unit that recognizes microbial carbohydrates. These components show structural similarities with the corresponding components of the classical pathway, the C1q-associated proteases, C1r and C1s⁴, and C1q³⁹, the antibody-recognizing unit. Here we present a new, phylogenetically highly conserved member of the MBL complex, MASP-3. We show that two different MBL/MASP complexes, MBL-cl and MBL-cll, can initiate complement activation. MBL-cl contains MASP-1 and MAp19 in association with MBL-1, the smallest MBL oligomer, and activates C3 directly, while MBL-cII contains MASP-2 in association with MBL-II and generates the C3 convertase, C4bC2b. MASP-3 is also associated with MBL-II and modulates MASP-2 activity.

Our studies on the MBL pathway led to the identification of a new lectin-associated protein. It was purified from plasma by sequential carbohydrate affinity chromatography and SDS-PAGE. N-terminal sequencing of the 42K protein suggested that it was a serine protease domain.

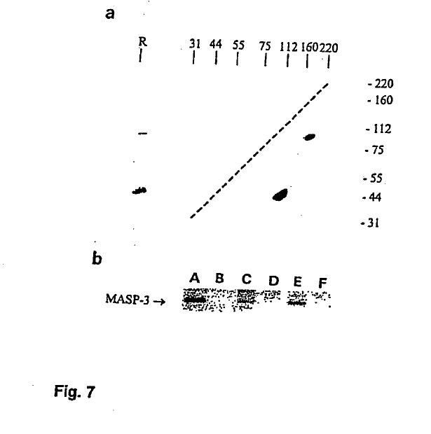

Antibody was raised against a synthetic peptide from the N-terminal sequence of the 42K protein. Two-dimensional SOS-PAGE and Western blofting using this antibody revealed that the presumed serine protease domain was derived from a protein of Mᵣ=105K. Before activation, the 105K protein forms a disulphide-linked dimer (FIG. 7a). Activation splits the 105K protein into 42K and 58K chains. The longer chain is not seen in the Western blots as it is not detected by the antibody used. This structure resembles the A and B chain structure of other serine proteases.

Analytical affinity procedures showed that the protein occurred in plasma as a complex with MBL (FIG. 7b). The protein thus bound to solid-phase anti-MBL antibody when MBL-sufficient serum was applied, but not when MBL-deficient serum was applied. When MBL was added to MBL-deficient serum, the protein again bound to the solid phase. The protein was accordingly termed MBL-associated serine protease-3, MASP-3.

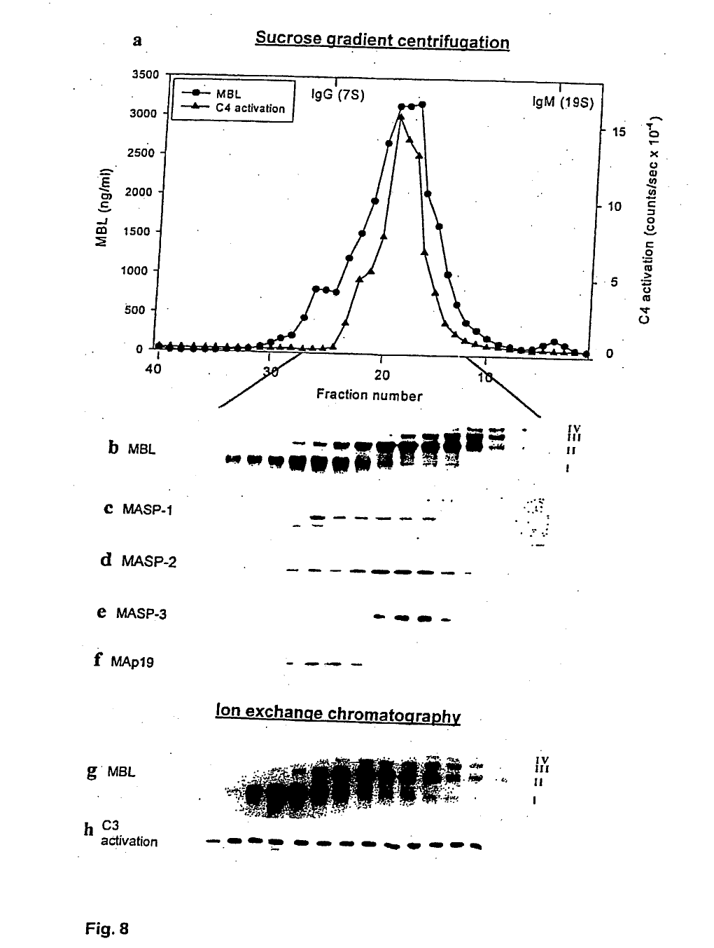

MBL complexes could be separated into different structural and functional forms by ion-exchange chromatography and sucrose gradient centrifugation. Four distinct MBL bands, MBL-I, II, III and IV, were revealed by non-reducing SDS-PAGE, with mobilities corresponding to approximate Mᵣs of 275K, 345K, 580K and 900K (FIG. 8b). On ion-exchange chromatography they were eluted in that order by increasing salt concentration, and on sucrose gradient centrifugation they showed sedimentation rates in the same order (FIG. 8). The presence of distinct MBL forms agrees with previous findings^{39,40}. Both fractionation methods showed MASP-1 and MAp19 to be associated largely with MBL-1, and MASP-2 and MASP-3 largely with MBL-II, although slightly staggered. The ability to activate C4, the first step in generating the C3 convertase, C4bC2b, coincided with the MBL-II complexes, MBL-cII (FIG. 8a). The MBL-I complexes (MBL-cI) were capable of activating C3 directly (FIG. 8h). This agrees with previous observations on the activity of isolated MASP-1^{4,41} and MASP-2²². It has also been shown that complexes composed of rMASP-2 and MBL can activate C4³⁰. Although the precise function of MASP-3 complexed with MBL was unknown, we examined the biological activity of MASP-3 using recombinant proteins produced in a mammalian expression system. This revealed a pronounced inhibitory activity of rMASP-3 on the activation of C4 by natural MBL complexes (FIG. 9a). The activity of rMBL-rMASP-2 complexes was also inhibited by rMASP-3 (FIG. 9b). To understand the biology of the MASPs it is important to realise that only a minor proportion of these proteases are associated with MBL in serum, as has been demonstrated for MASP-1 and MASP-2^{29,42}. By depleting serum of MBL complexes and analysing for residual MASP-3, we found the same to be true for this protein (not shown).

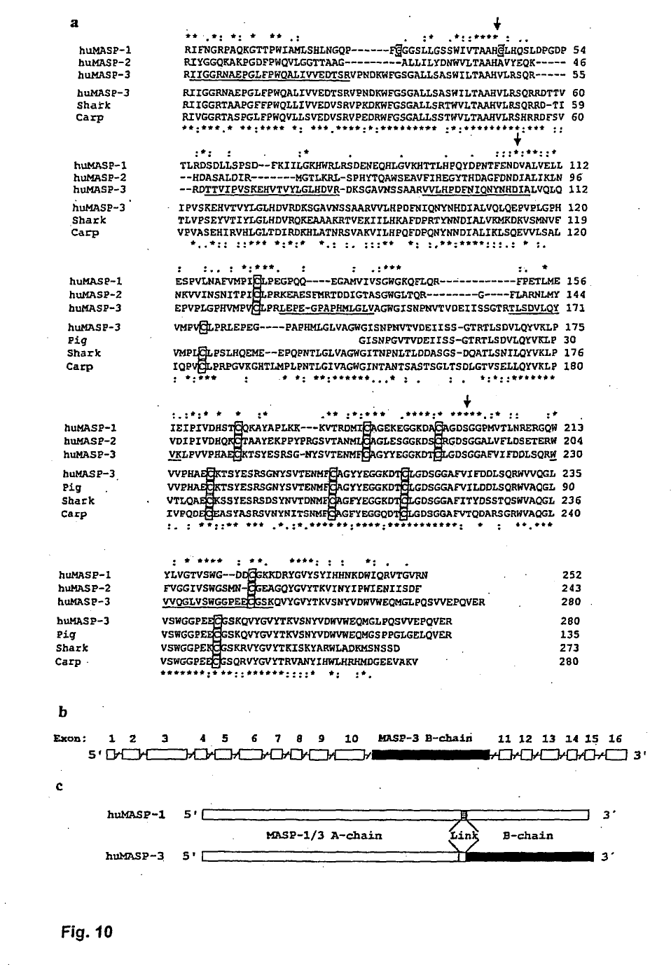

Further sequencing of MASP-3-derived peptides gave amino-acid sequences which were used to design and synthesise degenerated oligonucleotides. These were used for PCR amplification yielding a 174-base nucleotide fragment from liver cDNA. The deduced amino-acid sequence (FIG. 10a) classified the protein as a protease homologous to the B chains of MASP-1, MASP-2, C1r and C1s. At this stage a DNA sequence from the Human Genome Project was submitted to the data base (AC007920). The 230-kb sequence of random fragments contained the entire MASP-3 B-chain sequence as judged by comparison with the B chains of MASP-1, MASP-2, C1r and C1s. In addition, it contained the sequence for the ten exons encoding the MASP-1 A chain and the six exons encoding the MASP-18 chain. The relevant fragments were sorted on the basis of the published genome sequence of MASP-1⁴³, yielding the genomic structure shown schematically in FIG. 10b. The exon for the MASP-3 B chain is located between the exons encoding the MASP-1 A chain and the exons encoding the MASP-1 B chain. Further DNA sequences (AC068299, AC069069, AC034190 and AC046154) confirming this organisation have later entered the data bases. Primers were synthesized corresponding to the 5′ and 3′ ends of the MASP-3 B chain and used for PCR amplifications from genomic DNA and liver cDNA. Both reactions yielded DNA fragments which were cloned and sequenced and found to agree 100% with the sequence for the B chain in the data base. Thus, in contrast with the MASP-1 B chain but like the B chains of MASP-2, C1r and C1s, the MASP-3 B chain is encoded by a single exon. MASP-3, like MASP-2, C1r and C1s, lacks the histidine loop characteristic of MASP-1 and other trypsin-like proteases (FIG. 10a).

Cloning of MASP-3 cDNA from a human liver library revealed a transcription product composed of a common MASP-113 A chain and a unique MASP-3 B chain. This structure was confirmed by PCR on human liver cDNA using a primer pair corresponding to a sequence from exon 9 of the MASP-1 A chain and a sequence from the MASP-3 B chain (FIG. 10b). The last domain of the A chain is encoded by exons 9 and 10. Exon 10 is followed by an intron and the exon encoding the MASP-3 B chain. The largest clone, encoding full-length MASP-3 (pMASP-3; 4.1) comprises 3595 bp starting with a 5′ untranslated region of 90 bp, followed by an open reading frame (ORF) of 2184 bp and a 3′ untranslated region of 1321 bp, ending with a poly-A tail. The nucleotide sequence of pMASP-3; 4.1 has been deposited in GenBank (accession number AF 284421). The amino-acid sequences of the sequenced peptides were identified in the sequence deduced from the clone (FIG. 10a). The ORF encodes a polypeptide chain of 728 amino acids, including a signal peptide of 19 residues. Three N-glycosylation sites are found in the B chain and four in the A chain. Omitting the signal peptide, the calculated Mᵣ is 81,873 as compared with 105K observed on SDS-PAGE. The calculated isoelectric point is 5.02, and the molar extinction coefficient at 280 nm is 121,610 (absorbance of 1 g/l=1.49). The alternative splicing site was shown to be situated immediately after exon 10. The open reading frame of the B chain starts with a 42-bp untranslated sequence followed by the codons for the 14 residue link region. This link region precedes the activation site where the split between the A and B chains takes place (FIG. 10c). Antibody raised against a peptide representing the 20 N-terminal residues of the MASP-1 A chain recognized MASP-3 in Western blots as identified in parallel by the anti-MASP-3 B-chain antibody and by an antibody raised against a peptide representing the MASP-3 link region (not shown), thus identifying the MASP-3 protein as a product arising from alternative splicing.

Data-base searches revealed the homology of the MASP-3 B chain with sequences logged for shark and carp MASP243(FIG. 10a). The sequence identities are more than 60%, whereas those between human MASP-3 B chain and human MASP-1 and MASP-2 B chains are only 37% and 38%, respectively. Lamprey MASP shares a number of structural features with shark and carp MASP20. Although the sequence identity between lamprey MASP and human MASP-3 B chain is only 38%, we propose that the shark, carp and lamprey proteins are homologues of MASP-3.

A sequence logged for porcine DNA shows 93% identity with human MASP-3 B chain (FIG. 10a). This is an unusual degree of conservation in proteases, in which the constraint on individual amino-acid residues outside the catalytic centre is much less than for conserved structural proteins such as histones.

These results produce a clearer picture of the MBL complexes and the MBL pathway. There are distinct types of complexes: MBL-cI, which contains MASP-1 and MAp 19 and provides for direct activation of C3, and MBL-cII, which contains MASP-2 and activates C3 via the formation of the C3 convertase C4bC2b. MASP-3 is also associated with MBL-cII. rMASP-3-showed a modulating activity on complement activation. MASP-3 reveals interesting characteristics in its own right by representing a translation product of alternatively spliced RNA transcribed from the single gene encoding both MASP-1 and MASP-3. Phylogenetically the MASP-3 B chain is unusually highly conserved.

Methods

MBL Complexes

MBL complexes were purified by affinity chromatography on mannan-Sepharose in the presence of enzyme inhibitors, and were eluted with mannose-containing buffer⁴⁴.

Sucrose gradient centrifugation was performed by applying 100 μl MBL complex or 30 μl serum samples diluted with 70 μl Tris-buffered saline (TBS) to 11-ml sucrose gradients (10-30%) in TBS containing 5 mM CaCl₂ and 50 μg/ml human serum albumin and centrifuging at 35,000 rpm at 4° C. for 24 h in a Beckman L8-M centrifuge with a Sorval TST 41.14 rotor. Fractions of 0.3 ml were collected and the positions of IgG, IgM and MBL sedimentation peaks determined by time-resolved immunofluorometric assays (TRIFMA)²⁹.

For ion-exchange chromatography, MBL complexes were dialysed against 20 mM Tris/HCl, pH 7.8, containing 50 mM NaCl and 10 mM CaCl₂, and fractionated on a 1-ml Mono Q column (Arnersham-Pharmacia) with an NaCl gradient to 0.5 M. Fractions of 0.5 ml were collected and analysed for MBL by TRIFMA.

Fractions were also analysed by SDS-PAGE Western blotting against anti-MBL (Statens Serum Institut, Copenhagen, Denmark), anti-MASP-1²², anti-MASP-2²⁹ or anti-MASP-3 antibodies. Anti-MASP-3 antibody was raised against a peptide representing the first 19 amino-acid residues of the 42K chain by the method described. The blots were treated with horse radish peroxidase-labelled secondary antibody (Dako, Glostrup, Denmark) followed by enhanced chemiluminescence reagent (Pierce) and exposure to X-ray film. Markers for calculating Ms were from BioRad (“Precision Standards”), α2M and IgM (Sigma).

Amino-Acid Sequencing

A lectin preparation purified from plasma²² was subjected to SDS-PAGE, transferred to a PVDF membrane and stained with Coomassie Brilliant Blue. The 42K band was cut out and subjected to sequencing on an Applied Biosystems protein sequencer.

Peptides were prepared by tryptic digestion of the 42K band from a Coomassie-Blue-stained SDS-PAGE gel, fractionated by reverse phase chromatography and the peptides in the major peaks were sequenced.

C3 Activation

The ability of the MBL complexes in various fractions to activate C3⁴¹ was assessed by incubating 50 μl samples of fractions from ion-exchange chromatography with 50 ng purified C322 in 20 μl TBS at 37° C. for 2 h before analysing the digest by SDS-PAGE Western blotting using biotinylated anti-C3 antibody and avidin-peroxidase for development.

C4 activation

Activation of C4 was assessed by incubating samples at 4° C. in microtitre wells coated with mannan, followed by incubation at 37° C. with purified C4⁴⁵ and development with Eu-labelled monoclonal anti-C4 antibody2⁹.

MASP-3 cDNA and rMASP-3

PCR was performed on human liver cDNA (Clontech) using degenerated sense and antisense primers derived from the amino-acid sequences WQALIVVE and EHVTVYL, respectively. The PCR was carried out with annealing at 48° C. for 30 cycles using the long expand PCR system from Boehringer Mannheim. The resulting 174-bp PCR product was cloned into an E. coli plasmid (2.1-TOPO, InVitrogen) and the nucleotide sequence of the insert determined. By BLAST, this sequence identified a genomic fragment of 230 kb made up by random fragments (AC007917). Specific primers were used to obtain two cDNA clones (pMASP-3; 4.1 and pMASP-3; 3.0) in the pEAK8 vector (Pangene, Calif.). The inserts contained an open reading frame of 2163 bp encoding full length MASP-3.

Synthesis of rMASP-3 was accomplished by a procedure reported earlier³⁰. In brief, human embryonic kidney cells expressing the Epstein-Barr nuclear antigen (HEK 293EBNA, InVitrogen) were transfected with the pEAKS8pMASP-3; 4.1 construct and cultured in RPMI-1640 supplemented with insulin, transferrin and selenium (GibcoBRL). The culture supernatant was harvested after 6 d. A control was prepared by incubating the HEK 293EBNA cells with calcium phosphate precipitate without the construct.

1. A substantially pure mannan-binding lectin associated serine protease-3 (MASP-3) polypeptide, wherein said polypeptide comprises either
i) an amino acid sequence identified as SEQ ID NO 5 or a functional equivalent thereof comprising an amino acid sequence at least 85% identical to SEQ ID NO 5; or
ii) an amino acid sequence identified as SEQ ID NO 1 or a functional equivalent thereof comprising an amino acid sequence at least 85% identical to SEQ ID NO 1; or
iii) an amino acid sequence identified as SEQ ID NO 2 or a functional equivalent thereof comprising an amino acid sequence at least 50% identical to SEQ ID NO 2.

2. The polypeptide according to claim 1, said polypeptide being conjugated to a label or toxin.

3. The polypeptide according to any of the preceding claims, having a molecular mass of about 110 kDa under non-reducing conditions on an SDS-PAGE.

4. The polypeptide according to claim 3, said polypeptide containing the sequence Identified as SEQ ID NO 5.

5. The polypeptide according to any of the preceding claims, having a molecular mass of about 48 kDa under reducing conditions on an SDS-PAGE.

6. The polypeptide according to claim 5, said polypeptide containing the sequence identified as SEQ ID NO 5.

7. The polypeptide according to claim 1, said polypeptide having serine protease activity.

8. The polypeptide according to any of the preceding claims, said polypeptide being capable of MASP-3 activity in an in vitro assay for MBL pathway of complement function.

9. The polypeptide according to any of the preceding claims, said polypeptide being capable of competitively inhibiting MASP-3 serine protease activity.

10. The polypeptide according to claim 1 or a polypeptide comprising a fragment of the polypeptide of SEQ ID NO:1, SEQ ID NO:2 or SEQ ID NO:5, said polypeptide being a competitive inhibitor of complexing of MBL/MASP-3.

11. An Isolated nucleic acid molecule encoding the polypeptide of any of the claims 1 to 10, the molecule comprising a nucleotide sequence encoding a polypeptide having sequence that is at least 50% identical to the sequence of SEQ ID NO:1 or 2, or at least 85% identical to the sequence of SEQ ID NO: 5.

12. The isolated nucleic acid sequence according to claim 11, encoding a mannan-binding lectin associated serine protease-3 (MASP-3), wherein the nucleic acid comprises a sequence at least 85% identical to SEQ ID NO:3.

13. The isolated nucleic acid sequence according to claim 11, encoding a mannan-binding lectin associated serine protease-3 (MASP-3), said nucleic acid sequence being at least 85% identical to SEQ ID NO:4.

14. A nucleic acid vector comprising the nucleic acid molecule of any of the claims 11 to 13.

15. The nucleic acid vector of claim 14, wherein said vector is an expression vector.

16. The vector of claim 15, further comprising a regulatory element.

17. A cell comprising a vector as defined in any of claims 14 to 16.

18. A cell comprising a nucleic acid sequence as defined in any of claims 11 to 13.

19. The cell according to any of claims 17 to 18 being selected from a yeast cell, or a bacteria cell.

20. An antibody produced by administering a MASP-3 polypeptide, or part of a MASP-3 polypeptide, or DNA encoding a MASP-3 polypeptide, as defined in any of the claims 1-10 to an animal with the aim of producing antibody.

21. An antibody that selectively binds to a MASP-3 polypeptide as defined in any of claims 1 to 10.

22. The antibody according to any of claims 20 and 21, wherein said antibody is a monoclonal antibody or a genetically engineered antibody or an antibody fragment.

23. The antibody according to any of claims 20 to 22, said antibody being coupled to a compound comprising a detectable marker.

24. A compound capable of Inhibiting the complex formation of MBL and MASP-3, wherein said compound comprises a polypeptide as defined in any of claims 1-10.

25. A compound capable of inhibiting the complex formation of MBL and MASP-3, wherein said compound comprises an antibody as defined in any of claims 20 to 23.

26. A compound capable of disrupting the complex formation of MBL and MASP-3, wherein said compound comprises a polypeptide as defined in any of claims 1-10.

27. A compound capable of disrupting the complex formation of MBL and MASP-3, wherein said compound comprising an antibody as defined in any of claims 20 to 23.

28. A compound capable of competitively inhibiting serine protease activity of MASP-3 or a fragment thereof, said compound comprising a polypeptide as defined in any of claims 1-10.

29. A compound capable of competitively inhibiting serine protease activity of MASP-3 or a fragment thereof, said compound comprising an antibody as defined in any of claims 20 to 23.

30. A pharmaceutical composition comprising the polypeptide as defined in any of the claims 1-10, or an antibody as defined in any of the claims 20 to 23, or a compound as defined in any of the claims 24 to 29.

31. A method for detecting mannan-binding lectin associated serine protease-3 (MASP-3) in a biological sample, said method comprising:
(a) obtaining a biological sample;
(b) contacting said biological sample with a MASP-3 polypeptide specific binding partner that specifically binds MASP-3; and
(c) detecting said complexes, if any, as an indication of the presence of mannin-binding lectin associated serine protease-3 in said sample.

32. The method according to claim 31, in which the specific binding partner is an antibody according to any of the claims 20 to 23.

33. The method according to claim 31, wherein the specific binding partner is a mannan-binding lectin (MBL).

34. A method for determing the activity of MASP-3, said method comprising an assay for MASP-3 activity, comprising the steps of
a) applying a sample comprising MBL/MASP-2 complexes to a solid phase obtaining a bound complexes,
b) applying a predetermined amount of MASP-3 to the bound complexes
c) applying at least one complement factor to the complexes,
d) detecting the amount of cleaved complement factors,
e) correlating the amount of cleaved complement factors to the MASP-3 amount, and
f) determining the activity of MASP-3.

35. The method according to claim 34, wherein the solid phase is a mannan coating.

36. The method according to any of the preceding claims 34 to 35, wherein the at least one complement factor is a complement factor cleavable by the MBL/MASP-2 complex.

37. The method according to any of the preceding claims 34 to 36, wherein the at least one complement factor is selected from C3, C4, and C5, preferably C4.

38. The method according to any of the preceding claims 34 to 37, wherein the cleaved complement factor is detected by means of antibodies directed to the complement factor.

39. The method according to any of the preceding claims 34 to 38, wherein activation of the classical complement pathway is inhibited.

40. The method according to claim 39, wherein the activation is inhibited by conducting the assay at high ionic strength.

41. The method according to claim 40, wherein the salt concentration is in the range of from 0.3 M to 10 M, such as from 0.5 M to 5 M, such as from 0.7 M to 2 M, such as from 0.9 M to 2 M, such as about 1.0 M.

42. The method according to claim 28, wherein the salt is selected from NaCl, KCl, MgCl₂, CaCl₂, Nal, KCl, MgI₂, CaI₂, from NaBr, KBr, MgBr₂, CaBr₂, Na₂S₂O₃, (NH₄)₂SO₄, and NH₄HCO₃.

43. The method according to any of the claims 34 to 42 for quantitative assay of MASP-3 or MASP-3 activity in biological samples.

44. A method for detecting MASP-3 nucleic acid expression, comprising detecting RNA having a sequence encoding a MASP-3 polypeptide by mixing the sample with a nucleic acid probe that specifically hybridizes under stringent conditions to the nucleic acid as defined in any of claims 11 to 13.

45. A method for treating patients deficient in MASP-3 by administering to the patient the polypeptide as defined in any of claims 1 to 10.

46. A method for treating patients deficient in MASP-3 by administering to the patient nucleic acid as defined in any of claims 11 to 13.

47. A method for inhibiting the activity of MASP-3 by administering to the subject a compound that inhibits expression or activity of MASP-3.

48. The method of claim 47 in which the compound is a MASP-3 anti-sense nucleic acid sequence.

49. The method of claim 47 comprising administering a compound that inhibits complexing of MBL and MASP-3.

50. The method of claim 49, wherein the compound is as defined by any of the claims 24 to 29.

51. An assay for polymorphisms in the nucleic acid sequence encoding MASP-3.

52. A method of detecting the presence of MASP-3-encoding nucleic acid in a sample, comprising mixing the sample with at least one nucleic acid probe capable of forming a complex with MASP-3-encoding nucleic acid under stringent conditions, and determining whether the probe is bound to sample nucleic acid.

53. A nucleic acid probe capable of forming a complex with MASP-3-encoding nucleic acid under stringent conditions.

54. The nucleic acid probe according to claim 53, being a nucleic acid sequence capable of hybridizing to a nucleic acid sequence identical to SEQ ID NO 4.

55. The nucleic acid probe according to claim 53 to 54, being an anti-sense nucleic acid with respect to a nucleic acid sequence encoding MASP-3.

56. An assay for polymorphisms in the polypeptide sequence comprising MASP-3 or its precursor.

57. A method for diagnosing a disorder associated with aberrant expression of MASP-3, comprising obtaining a biological sample from a patient and measuring MASP-3 expression in said biological sample, wherein increased or decreased MASP-3 expression in said biological sample compared to a control indicates that said patient suffers from a disorder associated with aberrant expression of MASP-3.

58. A method for diagnosing a disorder associated with aberrant activity of MASP-3, comprising obtaining a biological sample from a patient and measuring MASP-3 activity in said biological sample, wherein increased or decreased MASP-3 activity in said biological sample compared to a control indicates that said patient suffers from a disorder associated with aberrant activity of MASP-3.

59. The use of a polypeptide as defined in any of the claims 1-10 for preparation of a pharmaceutical composition.

60. The use according to claim 59, wherein the pharmaceutical composition is capable of being administered parenterally, such as intramusculary, intravenously, or subcutaneously.

61. The use according to claim 59, wherein the pharmaceutical composition is capable of being administered orally.

62. The use according to any of the claim 59 to 61, wherein the pharmaceutical composition is suitable for the treatment of MASP-3 deficiency.

63. The use according to any of the claim 59 to 61, wherein the pharmaceutical composition is suitable for the treatment of immunesystem diseases, or of recoxygenated ischemic tissue.

64. The use of a compound as defined in any of the claims 24 to 29 for preparation of a pharmaceutical composition.

65. The use according to claim 64, wherein the pharmaceutical composition is capable of being administered parenterally, such as intramusculary, intravenously, or subcutaneously.

66. The use according to claim 64, wherein the pharmaceutical composition is capable of being administered orally.

67. The use according to any of the claim 64 to 66, wherein the pharmaceutical composition is suitable for the treatment of aberrant MASP-3 activity.

68. The use according to any of the claim 64 to 66 wherein the pharmaceutical composition is suitable for the treatment of infections, cancer, MBL-deficiency, disorders of the immunesystem and reproductive system.


SEQUENCE LISTING (26 sequences)

SEQ ID NO: 1 - protein, 20 aa, Homo sapiens (fig. 5, SEQ ID NO 1)
IIGGRNAEPGLFPWQALIVV

SEQ ID NO: 2 - protein, 58 aa, Homo sapiens (fig. 5, SEQ ID NO 2)
WQALIVVEDTSRVPNDKWFGSGALLSASWILTAAHVLRSQRRDTTVIPVSKEHVTVYL

SEQ ID NO: 3 - DNA, 174 bp, Homo sapiens (fig. 5, SEQ ID NO 3)
tggcaggccctgatagtggtggaggacacttcgagagtgccaaatgacaagtggtttgggagtggggccctgctctctgcgtcctggatcctcacagcagctcatgtgctgcgctcccagcgtagagacaccacggtgataccagtctccaaggagcatgtcaccgtctacctg

SEQ ID NO: 4 - DNA, 3895 bp, Homo sapiens (SEQ ID NO 4 and NO 5)
  CDS (91)..(2277): SEQ ID NO 5
attccggcacagggacacaaacaagctcacccaacaaagccaagctgggaggaccaaggccgggcagccgggagcacccaaggcaggaaaatgaggtggctgcttctctattatgctctgtgcttctccctgtcaaaggcttcagcccacaccgtggagctaaacaatatgtttggccagatccagtcgcctggttatccagactcctatcccagtgattcagaggtgacttggaatatcactgtcccagatgggtttcggatcaagctttacttcatgcacttcaacttggaatcctcctacctttgtgaatatgactatgtgaaggtagaaactgaggaccaggtgctggcaaccttctgtggcagggagaccacagacacagagcagactcccggccaggaggtggtcctctcccctggctccttcatgtccatcactttccggtcagatttctccaatgaggagcgtttcacaggctttgatgcccactacatggctgtggatgtggacgagtgcaaggagagggaggacgaggagctgtcctgtgaccactactgccacaactacattggcggctactactgctcctgccgcttcggctacatcctccacacagacaacaggacctgccgagtggagtgcagtgacaacctcttcactcaaaggactggggtgatcaccagccctgacttcccaaacccttaccccaagagctctgaatgcctgtataccatcgagctggaggagggtttcatggtcaacctgcagtttgaggacatatttgacattcaggaccatcctgaggtgccctgcccctatgactacatcaagatcaaagttggtccaaaagttttggggcctttctgtggagagaaagccccagaacccatcagcacccagagccacagtgtcctgatcctgttccatagtgacaactcggcagagaaccggggctggaggctctcatacagggctgcaggaaatgagtgcccagagctacagcctcctgtccatgggaaaatcgagccctcccaagccaagtatttcttcaaagaccaagtgctcgtcagctgtgacacaggctacaaagtgctgaaggataatgtggagatggacacattccagattgagtgtctgaaggatgggacgtggagtaacaagattcccacctgtaaaattgtagactgtagagccccaggagagctggaacacgggctgatcaccttctctacaaggaacaacctcaccacatacaagtctgagatcaaatactcctgtcaggagccctattacaagatgctcaacaataacacaggtatatatacctgttctgcccaaggagtctggatgaataaagtattggggagaagcctacccacctgccttccagagtgtggtcagccctcccgctccctgccaagcctggtcaagaggatcattgggggccgaaatgctgagcctggcctcttcccgtggcaggccctgatagtggtggaggacacttcgagagtgccaaatgacaagtggtttgggagtggggccctgctctctgcgtcctggatcctcacagcagctcatgtgctgcgctcccagcgtagagacaccacggtgataccagtctccaaggagcatgtcaccgtctacctgggcttgcatgatgtgcgagacaaatcgggggcagtcaacagctcagctgcccgagtggtgctccacccagacttcaacatccaaaactacaaccacgatatagctctggtgcagctgcaggagcctgtgcccctgggaccccacgttatgcctgtctgcctgccaaggcttgagcctgaaggcccggccccccacatgctgggcctggtggccggctggggcatctccaatcccaatgtgacagtggatgagatcatcagcagtggcacacggaccttgtcagatgtcctgcagtatgtcaagttacccgtggtgcctcacgctgagtgcaaaactagctatgagtcccgctcgggcaattacagcgtcacggagaacatgttctgtgctggctactacgagggcggcaaagacacgtgccttggagatagcggtggggcctttgtcatctttgatgacttgagccagcgctgggtggtgcaaggcctggtgtcctgggggggacctgaagaatgcggcagcaagcaggtctatggagtctacacaaaggtctccaattacgtggactgggtgtgggagcagatgggcttaccacaaagtgttgtggagccccaggtggaacggtgagctgacttacttcctcggggcctgcctcccctgagcgaagctacaccgcacttccgacagcacactccacattacttatcagaccatatggaatggaacacactgacctagcggtggcttctcctaccgagacagcccccaggaccctgagaggcagagtgtggtatagggaaaaggctccaggcaggagacctgtgttcctgagcttgtccaagtctctttccctgtctgggcctcactctaccgagtaatacaatgcaggagctcaaccaaggcctctgtgccaatcccagcactcctttccaggccatgcttcttaccccagtggcctttattcactcctgaccacttatcaaacccatcggtcctactgttggtataactgagcttggacctgactattagaaaatggtttctaacattgaactgaatgccgcatctgtatattttcctgctctgccttctgggactagccttggcctaatccttcctctaggagaagagcattcaggttttgggagatggctcatagccaagcccctctctcttagtgtgatcccttggagcaccttcatgcctggggtttctctcccaaaagcttcttgcagtctaagccttatcccttatgttccccattaaaggaatttcaaaagacatggagaaagttgggaaggtttgtgctgactgctgggagcagaatagccgtgggaggcccaccaagcccttaaattcccattgtcaactcagaacacatttgggcccatatgccaccctggaacaccagctgacaccatgggcgtccacacctgctgctccagacaagcacaaagcaatctttcagccttgaaatgtattatctgaaaggctacctgaagcccaggcccgaatatggggacttagtcgattacctggaaaaagaaaagacccacactgtgtcctgctgtgcttttgggcaggaaaatggaagaaagagtggggtgggcacattagaagtcacccaaatcctgccaggctgcctggcatccctggggcatgagctgggcggagaatccaccccgcaggatgttcagagggacccactccttcatttttcagagtcaaaggaatcagaggctcacccatggcaggcagtgaaaagagccaggagtcctgggttctagtccctgctctgcccccaactggctgtataacctttgaaaaatcattttctttgtctgagtctctggttctccgtcagcaacaggctggcataaggtcccctgcaggttccttctagctggagcactcagagcttccctgactgctagcagcctctctggccctcacagggctgattgttctccttctccctggagctctctctcctgaaaatctccatcagagcaaggcagccagagaagcccctgagagggaatgattgggaagtgtccactttctcaaccggctcatcaaacacactcctttgtctatgaatggcacatgtaaatgatgttatattttgtatcttttatatcatatgcttcaccattctgtaaagggcctctgcattgttgctcccatcaggggtctcaagtggaaataaaccctcgtggataaccaaaaaaaaaaaaaaaaaaaa

SEQ ID NO: 5 - protein, 728 aa, Homo sapiens (SEQ ID NO 4 and NO 5)
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIQDHPEVPCPYDYIKIKVGPKVLGPFCGEKAPEPISTQSHSVLILFHSDNSAENRGWRLSYRAAGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 6 - protein, 13 aa, Homo sapiens (fig. 4, td8)
EHVTVYLGLHDVR

SEQ ID NO: 7 - protein, 18 aa, Homo sapiens (fig. 4, td11)
  MISC_FEATURE (9)..(9): unsure
SVVQGLVSXGGPEEWGSK

SEQ ID NO: 8 - protein, 11 aa, Homo sapiens (fig. 4, td7)
  MISC_FEATURE (7)..(7): unsure
YEAEPGXYXGI

SEQ ID NO: 9 - protein, 25 aa, Homo sapiens (fig. 4, n-term)
IIGGRNAEPGLFPWQALIVVEDTSR

SEQ ID NO: 10 - protein, 16 aa, Homo sapiens (fig. 4, td13 and td14)
LEPEGPANIMNYLVDI

SEQ ID NO: 11 - protein, 15 aa, Homo sapiens (fig. 4, td13 and td14)
  MISC_FEATURE (9)..(9): unsure
VVLHPDFLXQLGNXA

SEQ ID NO: 12 - protein, 10 aa, Homo sapiens (fig. 4, td10)
TLSDVLQYVK

SEQ ID NO: 13 - protein, 8 aa, Homo sapiens (fig. 4, td4)
TTVIPVSK

SEQ ID NO: 14 - protein, 13 aa, Homo sapiens (fig. 4, td9)
EAANTLIADYVAQ

SEQ ID NO: 15 - protein, 20 aa, Homo sapiens (fig. 4, td18)
NAEPGLFPWQALIVVEDTSR

SEQ ID NO: 16 - protein, 284 aa, Homo sapiens (fig. 6, MASP-2)
MKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 17 - protein, 296 aa, Homo sapiens (fig. 6, MASP-1)
MLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLRDSDLLSPSDFKIILGKHWRLRSAENEQHLGVKHTTLHPQYDPNTFENVVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 18 - protein, 293 aa, Homo sapiens (fig. 6, C1r)
MQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 19 - protein, 296 aa, Homo sapiens (fig. 6, C1s) [sequence omitted]

SEQ ID NO: 20 - protein, 76 aa, Homo sapiens (fig. 6, MASP-3)
IIGGRNAEPGLFPWQALIVVEDTSRVPNDKWFGSGALLSASWILTAAHVLRSQRRDTTVIPVSKEHVTVYLGLHVR

SEQ ID NO: 21 - protein, 252 aa, Homo sapiens (fig. 10, huMASP-1)
RIFNGRPAQKGTTPWIAMLSHLNGQPFCGGSLLGSSWIVTAAHCLHQSLDPEDPTLRDSDLLSPSDFKIILGKHWRLRSAENEQHLGVKHTTLHPQYDPNTFENVVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 22 - protein, 243 aa, Homo sapiens (fig. 10, huMASP-2)
RIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 23 - protein, 280 aa, Homo sapiens (fig. 10, huMASP-3)
RIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 24 - protein, 135 aa, Pig (fig. 10)
GISNPGVTVDEIISSGTRTLSDVLQYVKLPVVPHAECKTSYESRSGNYSVTENMFCAGYYEGGKDTCLGDSGGAFVILDDLSQRWVAQGLVSWGGPEECGSKQVYGVYTKVSNYVDWVWEQMGSPPGLGELQVER

SEQ ID NO: 25 - protein, 273 aa, Shark (fig. 10)
RIIGGRTAAPGFFPWQLLIVVEDVSRVPKDKWFGSGALLSRTWVLTAAHVLRSQRRDTITLVPSEYVTIYLGLHDVRQKEAAAKRTVEKIILHKAFDPRTYNNDIALVKMKDKVSMNVFVMPLCLPSLHQEMEEPQPNTLGLVAGWGITNPNLTLDDASGSDQATLSNILQYVKLPVTLQAECKSSYESRSDSYNVTDNMFCAGFYEGGKDTCLGDSGGAFITYDSSTQSWVAQGLVSWGGPEKCGSKRVYGVYTKISKYARWLADKMSNSSD

SEQ ID NO: 26 - protein, 280 aa, Carp (fig. 10)
RIVGGRTASPGLFPWQVLLSVEDVSRVPEDRWFGSGALLSSTWVLTAAHVLRSHRRDFSVVPVASEHIRVHLGLTDIRDKHLATNRSVAKVILHPQFDPQNYNNDIALIKLSQEVVLSALIQPVCLPRPGVKGHTLMPLPNTLGIVAGWGINTANTSASTSGLTSDLGTVSELLQYVKLPIVPQDECEASYASRSVNYNITSNMFCAGFYEGGQDTCLGDSGGAFVTQDARSGRWVAQGLVSWGGPEECGSQRVYGVYTRVANYIHWLHRHMDGEEVAKV